(19) United States
(12) Patent Application Publication
(10) Pub. No.: US 2014/0018288 A1
(43) Pub. Date: Jan. 16, 2014

(54) X-DING-CD4 Peptide

(71) Applicant: St. Luke's-Roosevelt Hospital Center, New York, NY (US)

(72) Inventors: Malgorzata Simm, Pocono Lake, PA (US); Rakhee Sachdeva, New York, NY (US)

(21) Appl. No.: 13/943,337

(22) Filed: Jul. 16, 2013

Related U.S. Application Data
(60) Provisional application No. 61/672,033, filed on Jul. 16, 2012.

(51) Int. Cl. C07K 14/435

(52) U.S. Cl. CPC C07K 14/435
USPC 514/3.8; 530/350; 536/23.5; 435/69.1; 435/320.1; 435/455; 530/324; 530/327; 530/326; 530/330

(57) ABSTRACT

Here provided are a pharmaceutical composition containing an X-DING-CD4 peptide, a derivative of the X-DING-CD4 peptide, or a combination thereof a method for preventing or treating a pathological condition in a subject using the above pharmaceutical composition; and a process of making the above pharmaceutical composition. Also provided are isolated X-DING-CD4 cDNAs and isolated X-DING-CD4 peptides. Further provided are the composition and method for cell-based therapy using polynucleotides encoding X-DING-CD4 peptide, its derivative, or a combination thereof.

### CROSS REFERENCE TO PRIOR APPLICATION

This application claim priority to U.S. Provisional Application No. 61/672,033, filed on Jul. 16, 2012. The content of U.S. 61/672,033 is hereby incorporated by reference in its entirety.

### GOVERNMENTAL SUPPORT

This invention was made with Government support under grant 5RO1NS062649 awarded by National Institute of Neurological Disorders and Stroke, National Institutes of Health.

### FIELD OF INVENTION

The present invention relates to pharmaceutical compositions comprising an X-DING-CD4 peptide, or its derivatives and methods for preventing or treating pathological conditions, in particular, methods for preventing or treating viral infections and inflammatory conditions in human by using as active ingredients an X-DING-CD4 peptide, or its derivatives. The present invention further relates to isolated polynucleotides and isolated polypeptides.

### BACKGROUND

Viruses, capsules with genetic material inside, invade living cells and use those cells to multiply and reproduce themselves. Such reproduction may eventually kill the cells. Viral infection causes diseases such as acquired immune deficiency syndrome (AIDS)/HIV and hepatitis. Viral infections are generally hard to treat and there are not many antiviral medicines available. Antibiotics do not work for viral infections. Even for those infections for which there are antiviral medicines, human body may become resistant to the medicines. For example, three types of anti-HIV drugs are available: non-nucleotide reverse transcriptase inhibitors, nucleoside reverse transcriptase inhibitors, and protease inhibitors. However, about 80% of HIV patients are resistant to at least one HIV drugs; 45.5% of HIV patients are resistant to a double combination of nucleoside reverse transcriptase inhibitors and protease inhibitors; and 26% are resistant to a triple combination of non-nucleotide reverse transcriptase inhibitors, nucleoside reverse transcriptase inhibitors, and protease inhibitors (Tamalet et al., 2003). It is, therefore, desirable to have a new class of antiviral medicines so that patients resistant to current medicines may have more choices.

DING protein family members are a new group of highly conserved proteins found in prokaryotes and eukaryotes and throughout the plant and animal kingdoms (Adams et 2002: Belenky et al., 2003; Berna et al. 2007; Bema et al., 2002; Bema et al. 2009; Bernier and Berna, 2001; Bush et al. 1998; Collombet et al. 2010; Darbinian-Sarkissian et al., 2006; Darbinian et al., 2009; Diemer et al. 2008; Hain et al., 1996). They have diverse functions in cells and interestingly, some of these activities are directed to cellular protection from pathogen invasion (Amini et al., 2009; Berna et al., 2007; Berna et al., 2009; Bernier and Berna, 2001).

Native X-DING-CD4 (extracellular DING from CD4+ T cells), which we recently purified to homogeneity and functionally characterized in our laboratory (Lesner et al., 2009), is a new member of innate protective DING molecules produced by human cells. Native X-DING-CD4 protein is soluble and has two isomeric forms distinguished by rare methylation of glutamic acid (Leaner et al., 2009). Native X-DING-CD4 protein may act through receptor independent pathway by blocking nuclear factor of kappa light polypeptide gene enhancer in B-cells 1 (NF-κB) binding to HIV long terminal repeat (LTR) (Lesner et al., 2005; Lesner et al., 2009: Li et al., 2007; Simm et al., 2002).

### SUMMARY OF THE INVENTION

In one aspect, the present invention is directed to a pharmaceutical composition for preventing or treating a pathological condition in a subject. The composition contains comprising a pharmaceutically effective amount of an X-DING-CD4 peptide having an amino acid sequence set forth in SEQ ID NO: 25, a derivative of the X-DING-CD4 peptide, or a combination thereof.

In another aspect, the present invention is directed to a method for preventing or treating a pathological condition in a subject by the administration of a pharmaceutically effective amount of an X-DING-CD4 peptide having an amino acid sequence set forth in SEQ ID NO: 25, a derivative of the X-DING-CD4 peptide, or a combination thereof.

In some embodiment of the above pharmaceutical composition or method, the pathological condition may be a viral infection or a retroviral infection such as an HIV, e.g., HIV-1, infection.

In some embodiments of the above pharmaceutical composition or method, the derivative of the peptide set forth in SEQ ID NO: 25 can be polypeptides having amino acid sequences set forth in SEQ ID NOS: 1-15. In other embodiments, the therapeutically active derivative of the X-DING-CD4 peptide has at least about 85% sequence identity to the X-DING-CD4 peptide.

In some embodiments of the above pharmaceutical composition or method, the pharmaceutically effective amount of the X-DING-CD4 polypeptide or its derivative is from about 1 nM to about 200 nM, in particular from about 8 nM to about 150 nM, preferentially 100 nM. In some embodiments, the X-DING-CD4 polypeptide or its derivative can be used in a mammal, or more preferably in a human.

In a further aspect, the present invention is directed to an isolated complementary deoxyribonucleic acid (cDNA), which encodes an X-DING-CD4 peptide comprising the amino acid sequence set forth in SEQ ID NO: 25 or a derivative of the X-DING-CD4 peptide.

In some embodiments, the isolated cDNA may encode a polypeptide having amino acid sequences set forth in SEQ ID NOS: 1-15. In some embodiments, the cDNA may be cloned into a vector to form a plasmid which may be optionally placed in a host cell. For one instance, the plasmid vector may be pET-28a and the host may be a bacteria E. coli BL21. For another instance, the plasmid vector may be pcDNA 3.1 vector and the host may be a mammalian cell, e.g., Human Embryonic Kidney 293T cells.

In still further aspect, the present invention is directed to an isolated cDNA comprising the nucleic acid sequence set forth in SEQ ID NO: 16 or 26, or a polynucleotide that is complementary to the polynucleotide set forth in SEQ ID NO: 16 or 26.

In a further aspect, the present invention is directed to an isolated polypeptide comprising an amino acid sequence having at least 85% identity to SEQ ID NO: 25 and a derivative of the above polypeptide. In some embodiments, the glutamic acid at position 68 in SEQ ID No: 25 is methylated. In other embodiments, the isolated polypeptide derivative is a polypeptide having amino acid sequences set forth in SEQ ID NOS: 1-15.

In some embodiments, the isolated polypeptide has the ability to block a NF-κB-mediated biological pathway, e.g., a pathway that regulates a viral gene transcription such as a HIV-1 LTR gene transcription. As such, in some embodiments, the isolated polypeptide may inhibit a viral infection, e.g., a HIV infection.

In some embodiments, the isolated polypeptide has the ability to block a NF-κB-mediated biological pathway that controls an inflammatory reaction, e.g., a lipopolysaccharide-induced inflammatory reaction. As such, in some embodiments, the isolated polypeptide may inhibit an inflammatory reaction, e.g., a lipopolysaccharide-induced inflammatory reaction. In some instances, the lipopolysaccharide is from a bacteria, such as a Salmonella typhimurium, Shigella flexneri, or Camplylobacter jejuni.

In still a further aspect, the present invention is directed to a process for producing a polypeptide by culturing the host cell that contains a plasmid encompassing an isolated cDNA that encodes an X-DING-CD4 peptide having the amino acid sequence set forth in SEQ ID NO: 25 or a derivative of the X-DING-CD4 peptide under conditions sufficient for the production of the polypeptide. In some instance, the process further comprises recovering the polypeptide so produced. The recovery method may employ ion-exchange chromatography, e.g., a Ni-affinity column.

In still another aspect, the present invention is directed to a composition for a cell-based therapy. The composition includes a cDNA encoding an X-DING-CD4 peptide having an amino acid sequence set forth in SEQ ID NO: 25, a derivative of the X-DING-CD4 peptide, or a combination thereof. In some embodiments, the cDNA is inserted in cell-base therapy vector, e.g., retroviral vector or an adenovirus vector. In some embodiments, the cell-based therapy is to prevent or treat viral infection such as a HIV infection or to prevent or treat inflammation reactions.

In still a further aspect, the present invention is directed to a method of a cell-based therapy by administrating into a cell a cDNA encoding an X-DING-CD4 peptide having an amino acid sequence set forth in SEQ ID NO: 25, or a derivative of the X-DING-CD4 peptide. In some embodiments, the cDNA is inserted in cell-base therapy vector, e.g., retroviral vector or an adenovirus vector. In some embodiments, the cell-based therapy is to prevent or treat viral infection such as a HIV infection or to prevent or treat inflammation reactions.

### BRIEF DESCRIPTION OF THE DRAWINGS

FIG. 1 shows an alignment of X-DING-CD4 peptide (SEQ ID NO: 25) to three DING protein family members: PfluDING from Pseudomonas fluorescens, pDING (p38SJ) from Hypericum perforatum, and human phosphate binding protein (HPBP) from human plasma. Accession numbers for each protein are indicated between bars, identical residues are highlighted with black. The sequence alignment was done using Bioedit software.

FIG. 2 shows a graph depicting the results of testing the biological activity of rX-DING-CD4^{pep} in blocking HIV-1 LTR promoter transcription by rapid suppression assay (RSA). rX-DING-CD4^{pep} refers to recombinant X-DING-CD4 peptide with the amino acid sequence set forth in SEQ ID NO: 1.

FIG. 3 shows a graph depicting the results of testing the biological activity of rX-DING-CD4^{pep} in inhibiting HIV-1 replication. rX-DING-CD4^{pep} refers to recombinant X-DING-CD4 peptide with the amino acid sequence set forth in SEQ ID NO: 1.

FIG. 4 shows a graph depicting the results of testing rX-DING-CD4^{pep} toxicity in vivo. nX-DING-CD4 refers to native form of X-DING-CD4 protein purified through ICX chromatography from the extracellular compartment of HIV-1 resistant T-cells. rX-DING-CD4^{pep} refers to recombinant X-DING-CD4 peptide with the amino acid sequence set forth in SEQ ID NO: 1.

FIG. 5 shows a graph depicting the results of assaying the biological activity of X-DING-CD4 peptide derivatives in blocking HIV-1 LTR promoter transcription by rapid suppression assay (RSA). The derivatives are listed in Table 1 having amino acid sequences set forth in SEQ ID NOS: 1-15.

FIGS. 6A and 6B show charts depicting EcoHIV Tat and Vif mRNA level determination in EcoHIV mice treated with rX-DING-CD4^{pep}, 0.9% saline control, or (HI)rX-DING-CD4^{pep}. RX-DING-CD4/EcoHIV indicates the group of animals subjected to treatments with rX-DING-CD4^{pep}. (HI)ᵣX-DING-CD4/EcoHIV indicates the group of animals subjected to treatments heat inactivated rX-DING-CD4^{pep}. EcoHIV depicts systems were animals were injected with 0.9% saline. Circles refer to EcoHIV Tat (FIG. 6A) and Vif (FIG. 6B) mRNAs; squares refer to Gag DNA. rX-DING-CD4^{pep} refers to recombinant X-DING-CD4 peptide with the amino acid sequence set forth in SEQ ID NO: 1.

FIG. 7 depicting the mechanism of how X-DING-CD4 protein functions in regulating HIV-1 LTR transcription in a cell. N referring to nucleus; NM referring to nucleus membrane; C referring to cytosol; M referring to cell membrane; X referring to extracellular space. The numbers −4 in the top-shadowed portion of the figure indicate function of the exogenous X-DING-CD4; while numbers 1-7 in the bottom portion of the figure indicate steps of activity of the endogenous X-DING-CD4.

FIG. 8 shows a photograph of a Western Blot depicting the compartmentalization of X-DING-CD4 proteins inside a cell. The three lanes are labeled Nuclei, Cell Membrane, and Cytosol, respectively, referring to three compartments inside a cell. P-nX-DING-CD4 referring to protein forms with phosphate polyatomic ion (PO₄³⁻); nX-DING-CD4 referring to protein forms present in nucleus without PO₄³⁻; n+cX-DING-CD4 referring to the protein forms present in both nucleus and cytosol; cX-DING-CD4 referring to the protein forms present only in cytosol; sX-DING-CD4 referring to the soluble protein form of about 39 kDa.

FIG. 9 shows a photograph of a Western Blot depicting the presence of X-DING-CD4/calcineurin complex in a cell. CnA referring to calcineurin A; CnB referring to calcineurin B; α-CnA referring to anti-CnA antibody; α-CnB referring to anti-CnB antibody; α-X-DING-CD4 referring to anti-X-DING-CD4 antibody (a mouse monoclonal to X-DING-CD4).

FIG. 10 shows a chart depicting the presence of NF-κB/LTR-X-DING-CD4 protein complex in a cell. PMA referring to phorbol 12-myristate 13-acetate; NF-κB referring to experimental results by using p65 NF-κB antibody; X-DING-CD4 referring to experimental results by using X-DING-CD4 antibody (a mouse monoclonal to X-DING-CD4).

FIG. 11 shows a chart depicting the inhibition of X-DING-CD4 activity by various phosphatase inhibitors. Tyr referring to tyrosine phosphatase inhibitors; Ser/Thr referring to serine/threonine phosphatase inhibitors. The inhibition activity was measured by determining the ability to block HIV-1 LTR transcription.

FIG. 12 shows a chart depicting the inhibition of X-DING-CD4 activity by various cellular uptake inhibitors. CPZ refers to Chromopromazine, an inhibitor of clatrin mediated endocytosis. CCD refers to cytochalasin D and CQ refers to chloroquine, both of which are endocytosis inhibitors. DMA refers to dimethyl amiloride, a macropinocytosis inhibitor. The numbers underneath the x-axis refer to the final concentrations of each inhibitor for treating the cells.

FIG. 13 shows representative images taken by confocal microscopy depicting the cellular uptake of sX-DING-CD4 by human fetal astrocytes. The localization of sX-DING-CD4 molecules were visualized by using antibodies to X-DING-CD4 (green) and Spectrin (red). Spectrin is used to mark the location of the cell membrane.

FIG. 14 shows a chart depicting the induction of X-DING-CD4 mRNA expression by a native X-DING-CD4 protein (“X-DING” in the chart) or a recombinant X-DING-CD4 peptide (“1-126 rX-DING” in the chart, with amino acid sequence set forth in SEQ ID NO: 1). The numbers underneath of x-axis refer to the concentration of agents used to treat the cells. For example, 200 ng/ml X-DING-CD4 peptide was used to treat the cells.

FIGS. 15A and 15B show charts depicting activities of synthetic X-DING peptides XD-1 (SEQ ID NO: 14) and XD-4 (SEQ ID NO: 15) in inducing IFN-α (FIG. 15A) and X-DING-CD4 (FIG. 15B) mRNA expression in 1G5 T cell line. nX-DING referring to native X-DING-CD4 protein; ¹⁻¹²⁶rX-DING referring to recombinant X-DING-CD4^{pep} (SEQ ID NO: 1).

FIGS. 16A and 16B show charts depicting the efficacy of DING family proteins to block HIV-1 LTR transcription as measured by RSA (FIG. 16A); and to block HIV-1 replication as measured by MAGI (FIG. 16B). The C3 peptide P16 was used as a negative control. The results are representative of at least three separate experiments. Dose versus response was calculated by GraphPad Prism version 5.00 for Windows. X-DING-CD4 refers to native X-DING-CD4 protein.

FIG. 17 shows a chart depicting inhibition effects of DING family proteins on HIV-1 replication in human PBLs. Replication of HIV-1 was assessed by measurements of HIV-1 p24 core antigen (bars, right axis) and the viability of cells was established by the dye exclusion method (lines, left axis). The results are representative of at least three separate measurements. X-DING-CD4 refers to native X-DING-CD4 protein.

### DETAILED DESCRIPTION OF THE INVENTION

The present invention is partly based on the discovery of a biologically active X-DING-CD4 peptide and an X-DING-CD4 cDNA that encodes the X-DING-CD4 peptide. The present invention is partly based on another discovery that recombinant and synthetic X-DING-CD4 peptides or their derivatives can inhibit viral infection and suppress inflammatory reactions.

As such, in one aspect, the invention is directed to isolated X-DING-CD4 peptide and its derivatives, and an isolated X-DING-CD4 cDNA encoding the X-DING-CD4 peptide and its derivatives. In one embodiment, the X-DING-CD4 peptide or its derivatives have amino acid sequences set forth in SEQ ID NO: 1-15, and 25. In another embodiment, the X-DING-CD4 cDNA has a polynucleotide sequence set forth in SEQ ID NO: 16 or SEQ ID NO: 26.

The phrase “isolated” means altered or removed from the natural state through the actions of a human being. For example, a polynucleotide is said to be “isolated” when it is substantially separated from contaminant polynucleotides that correspond or are complementary to genes other than the X-DING-CD4 gene or that encode polypeptides other than X-DING-CD4 gene product or derivatives thereof. A skilled artisan can readily employ nucleic acid isolation procedures as taught in this invention to obtain an isolated X-DING-CD4 polynucleotide in accordance with this invention. For another example, a peptide is said to be “isolated” when physical, mechanical or chemical methods are employed to remove the X-DING-CD4 peptide from cellular constituents that are normally associated with the peptide. A skilled artisan can readily employ purification methods as taught in this invention to obtain an isolated X-DING-CD4 peptide or its derivatives. Alternatively, an isolated peptide can be prepared by chemical means, for example, de novo synthesis.

The term “polynucleotide” means a polymeric form of nucleotides of at least 10 bases or base pairs in length, either ribonucleotides or deoxynucleotides or a modified form of either type of nucleotide, and is meant to include single and double stranded forms of DNA and/or RNA. A polynucleotide can be a recombinant polynucleotide, which is a polynucleotide that has been subjected to molecular manipulation in vitro.

The term “peptide” refers to a compound comprised of at least two amino acid residues covalently linked by peptide bonds or modified peptide bonds. Throughout the specification, standard three letter or single letter designations for amino acids are used. In the art, this term is often used interchangeably with “polypeptide” or “protein”. No limitation is placed on the maximum number of amino acids which can comprise a protein or peptide. The amino acids comprising the peptides or proteins described herein and in the appended claims are understood to be either D or L amino acids with L amino acids being preferred.

The amino acid comprising the peptides or proteins described herein can also be modified either by natural processes, such as posttranslational processing, or by chemical modification techniques which are well known in the art. Modifications can occur anywhere in a peptide, including the peptide backbone, the amino acid side-chains and the amino or carboxyl termini. It is understood that the same type of modification can be present in the same or varying degrees at several sites in a given peptide. Also, a given peptide can contain many types of modifications. Modifications may include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. See, for instance, Proteins—Structure and Molecular Properties, 2nd Ed., T. E. Creighton, W. H. Freeman and Company, New York, 1993 and Wold, F., Posttranslational Protein Modifications: Perspectives and Prospects, pgs. 1-12 in Posttranslational Covalent Modification Of Proteins, B. C. Johnson, Ed., Academic Press, New York, 1983; Seifter et al., “Analysis for protein modifications and nonprotein cofactors”, Meth Enzymol (1990) 182:626-646 and Rattan et al., “Protein Synthesis: Posttranslational Modifications and Aging”, Ann NY Acad Sci (1992) 663:48-62.

Derivatives of X-DING-CD4 peptide may include any naturally occurring or purposefully generated X-DING-CD4 peptide which is characterized by single or multiple amino acid truncations, substitutions, deletions, additions, or replacements. Unless otherwise indicated, the term “X-DING-CD4 peptide” is used herein to refer to the peptide having an amino acid sequence set fort in SEQ ID NO: 25. The derivatives of X-DING-CD4 peptide may include, but are not limited to (a) derivatives in which one or more amino acid residues of X-DING-CD4 peptide are substituted with conservative or non-conservative amino acids; (b) derivatives in which one or more amino acids are added; (c) derivatives in which one or more of the amino acids include a substituent group; (d) derivatives in which X-DING-CD4 peptide or a portion thereof is fused to another peptide (e.g., serum albumin or protein transduction domain); (e) derivatives in which one or more nonstandard amino acid residues (i.e., those other than the 20 standard L-amino acids found in naturally occurring proteins) are incorporated or substituted into the X-DING-CD4 peptide sequence; (f) derivatives in which one or more non-amino acid linking groups are incorporated into or replace a portion of X-DING-CD4 peptide; (g) derivatives in which X-DING-CD4 peptide is truncated into fragments of various sizes; (h) derivatives in which one or more amino acids in X-DING-CD4 peptide is modified as elaborated in the previous paragraph; and (i) a combination of any of the derivatives thereof. In some instances, the derivatives where changes of amino acids are made have at about 85% amino acid sequence identity to corresponding part in the X-DING-CD4 peptide. In other instances, the identity is above 90% and preferably above 95%. In still other instances, the identity is 100% and there were one or more amino acid modifications but no substitution, deletion, or insertion of amino acids.

In some embodiments, the invention is directed to primers and primer pairs, which allow the specific amplification of X-DING-CD4 polynucleotides of the invention or of any specific parts thereof, and probes that selectively or specifically hybridize to nucleic acid molecules of the invention or to any part thereof. Probes can be labeled with a detectable marker, such as, for example, a radioisotope, fluorescent compound, bioluminescent compound, a chemiluminescent compound, metal chelator or enzyme. Such probes and primers are used to detect the presence of an X-DING-CD4 polynucleotide in a sample and as a means for detecting a cell expressing an X-DING-CD4 peptide.

Examples of such probes include polynucleotides comprising all or part of the human X-DING-CD4 cDNA sequence set forth in SEQ ID NO: 26. Examples of primer pairs capable of specifically amplifying X-DING-CD4 mRNA are described in the Example 1. As will be understood by the skilled artisan, a great many different primers and probes can be prepared based on the sequences provided herein and used effectively to amplify and/or detect the X-DING-CD4 mRNA.

In some embodiments, the peptides in this invention are modified with penetrating agents. As used herein the phrase “penetrating agent” refers to an agent which enhances translocation of any of the attached peptide across a cell membrane. According to one embodiment, the penetrating agent is a peptide and is attached to the X-DING-CD4 peptide or its derivatives (either directly or indirectly) via a peptide bond.

Typically, peptide penetrating agents have an amino acid composition containing either relatively abundant positively charged amino acids such as lysine or arginine, or have sequences that contain an alternating pattern of polar/charged amino acids and non-polar, hydrophobic amino acids.

Examples of peptide penetrating agents include those set forth in SEQ ID NOs: 20-22. By way of non-limiting example, cell penetrating peptide (CPP) sequences may be used in order to enhance intracellular penetration. CPPs may include short and long versions of TAT (YGRKKRR—SEQ ID NO: 20 and YGRKKRRQRRR—SEQ ID NO: 21) and HD (RRQRR—SEQ ID NO: 22). However, the disclosure is not so limited, and any suitable penetrating agent may be used, as known by those of skill in the art.

The X-DING-CD4 peptides of the present invention may also comprise non-amino acid moieties, such as for example, hydrophobic moieties (various linear, branched, cyclic, polycyclic or heterocyclic hydrocarbons and hydrocarbon derivatives) attached to the peptides; non-peptide penetrating agents; various protecting groups, especially where the compound is linear, which are attached to the compound's terminals to decrease degradation. Chemical (non-amino acid) groups present in the compound may be included in order to improve various physiological properties such; decreased degradation or clearance; decreased repulsion by various cellular pumps, improve immunogenic activities, improve various modes of administration (such as attachment of various sequences which allow penetration through various barriers, through the gut, etc.); increased specificity, increased affinity, decreased toxicity and the like.

Attaching the amino acid sequence component of the peptides of the invention to other non-amino acid agents may be achieved, but is not limited to, by covalent linking; by non-covalent complexion, for example, by complexing to a hydrophobic polymer, which can be degraded or cleaved thereby producing a compound capable of sustained release; and by entrapping the amino acid part of the peptide in liposomes or micelles to produce the final peptide of the invention. The association may be by the entrapment of the amino acid sequence within the other component (liposome, micelle) or the impregnation of the amino acid sequence within a polymer to produce the final peptide of the invention.

In one embodiment, soluble X-DING-CD4 peptides may be linked with cholesterol to make therapeutic agents. The cellular uptake of this X-DING-CD4-cholesterol complex may be induced by HIV-1 entry. This is consistent with published evidence that HIV-1 infection causes hypercholesterolemia (Falasca et al., 2006) resulting in rapid uptake of lipoproteins carrying in the cholesterol particles (Meyer et al., 2012) and that human DING variant, the HPBP binds efficiently the HDL and IDL particles (Renault et al., 2006).

The peptides of the invention may be linear or cyclic (cyclization may improve stability). Cyclization may take place by any means known in the art. Where the compound is composed predominantly of amino acids, cyclization may be via N- to C-terminal, N-terminal to side chain and N-terminal to backbone, C-terminal to side chain, C-terminal to backbone, side chain to backbone and side chain to side chain, as well as backbone to backbone cyclization. Cyclization of the peptide may also take place through non-amino acid organic moieties comprised in the peptide.

In another aspect, the invention is a process of making X-DING-CD4 peptide and its derivatives. Peptides can be biochemically synthesized such as by using standard solid phase techniques. These methods include, but are not limited to, exclusive solid phase synthesis, partial solid phase synthesis methods, fragment condensation, classical solution synthesis. Solid phase polypeptide synthesis procedures are well known in the art and further described by John Morrow Stewart and Janis Dillaha Young, Solid Phase Polypeptide Syntheses (2nd Ed., Pierce Chemical Company, 1984).

Large scale peptide synthesis is described by Andersson Biopolymers 2000; 55(3):227-50. Synthetic peptides can be purified by preparative high performance liquid chromatography [Creighton T. (1983) Proteins, Structures and Molecular Principles. WH Freeman and Co. N.Y.] and the composition of which can be confirmed via amino acid sequencing.

Recombinant techniques may also be used to generate the peptides of the present invention. To produce a peptide of the present invention using a recombinant technique, a polynucleotide encoding the peptide of the present invention may be ligated into a nucleic acid expression vector, which comprises the polynucleotide sequence under the transcriptional control of a cis-regulatory sequence (e.g., promoter sequence) suitable for directing constitutive, tissue specific or inducible transcription of the polypeptides of the present invention in the host cells.

Recombinant DNA or RNA molecules containing an X-DING-CD4 polynucleotide, a fragment, or homologue thereof, include, but are not limited to phages, plasmids, phagemids, cosmids, YACs, BACs, as well as various viral and non-viral vectors well known in the art, and cells transformed or transfected with such recombinant DNA or RNA molecules. Methods for generating such molecules are well known. See, for example, Sambrook, J. et al., Molecular Cloning: A Laboratory Manual, 2d edition, Cold Spring Harbor Press, New York, 1989; Current Protocols in Molecular Biology. Ausubel et al., Eds., Wiley and Sons, 1995.

The term “homolog” refers to a molecule which exhibits homology to another molecule, by for example, having sequences of chemical residues that are the same or similar at corresponding positions.

In one embodiment, the X-DING-CD4 cDNA may be cloned into an expression vector. As used herein, the term “expression vector” refers to a vector containing a nucleic acid sequence coding for at least part of a gene product capable of being transcribed. In some cases, RNA molecules are then translated into a protein, polypeptide, or peptide. Expression vectors can contain a variety of control sequences, which refer to nucleic acid sequences necessary for the transcription and possibly translation of an operatively linked coding sequence in a particular host organism.

As such, the invention further provides a host-vector system comprising a recombinant DNA molecule containing an X-DING-CD4 polynucleotide, fragment, or homologue thereof within a suitable prokaryotic or eukaryotic host cell. Examples of suitable eukaryotic host cells include a yeast cell, a plant cell, or an animal cell, such as a mammalian cell or an insect cell (e.g., a baculovirus-infectible cell such as an Sf9 or HighFive cell). Examples of suitable mammalian cells include various prostate cancer cell lines such as DU145 and TsuPr1, other transfectable or transducible prostate cancer cell lines, primary cells (PrEC), as well as a number of mammalian cells routinely used for the expression of recombinant proteins (e.g., COS, CHO, 293, 293T cells). More particularly, a polynucleotide comprising the coding sequence of X-DING-CD4 peptide or a fragment, analog or homolog thereof can be used to generate X-DING-CD4 proteins or fragments thereof using any number of host-vector systems routinely used and widely known in the art.

A wide range of host-vector systems suitable for the expression of X-DING-CD4 proteins or derivatives thereof are available, see for example, Sambrook et al., 1989, supra; Current Protocols in Molecular Biology, 1995, supra). Preferred vectors for mammalian expression include but are not limited to pcDNA 3.1 myc-His-tag (Invitrogen) and the retroviral vector pSR.alpha.tkneo (Muller et al., 1991, MCB 11:1785). Using these expression vectors, X-DING-CD4 peptide can be expressed in several cell lines, including for example 293, 293T, rat-1, NIH 3T3 and TsuPr1. The host-vector systems of the invention are useful for the production of an X-DING-CD4 peptide or derivatives thereof. Such host-vector systems may also be employed to study the biological properties of X-DING-CD4 peptide and its derivatives.

Recombinant human X-DING-CD4 peptide and it derivatives may be produced by mammalian cells transfected with a construct including an X-DING-CD4 polynucleotide. For example, 293T cells can be transfected with an expression plasmid encoding X-DING-CD4 peptide or its derivative, an X-DING-CD4-related protein is expressed in the 293T cells, and the recombinant X-DING-CD4 peptide is isolated using standard purification methods (e.g., affinity purification using anti-X-DING-CD4 peptide antibodies). In another embodiment, an X-DING-CD4 cDNA is subcloned into the retroviral vector pSR.alpha.MSVtkneo and used to infect various mammalian cell lines, such as NIH 3T3, TsuPr1, 293 and rat-1 in order to establish X-DING-CD4^{pep} expressing cell lines. Various other expression systems well known in the art can also be employed. Expression constructs encoding a leader peptide joined in frame to an X-DING-CD4 peptide coding sequence can be used for the generation of a secreted form of the recombinant X-DING-CD4 peptide. For example, recombinant X-DING-CD4^{pep} can be produced in bacteria cells as detailed in Example 1.

Redundancy in the genetic code permits variation in X-DING-CD4 gene sequences. In particular, it is known in the art that specific host species often have specific codon preferences, and thus one can adapt the disclosed sequence as preferred for a desired host. For example, preferred analog codon sequences typically have rare codons (i.e., codons having a usage frequency of less than about 20% in known sequences of the desired host) replaced with higher frequency codons. Codon preferences for a specific species are calculated, for example, by utilizing codon usage tables known in the art.

Additional sequence modifications known to enhance protein expression in a cellular host may also be employed. These include elimination of sequences encoding spurious polyadenylation signals, exon/intron splice site signals, transposon-like repeats, and/or other such well-characterized sequences that are deleterious to gene expression. The GC content of the sequence is adjusted to levels average for a given cellular host, as calculated by reference to known genes expressed in the host cell. Where possible, the sequence is modified to avoid predicted hairpin secondary mRNA structures. Other useful modifications include the addition of a translational initiation consensus sequence at the start of the open reading frame, as described in Kozak, Mol. Cell. Biol., 9:5073-5080 (1989). Skilled artisans understand that the general rule that eukaryotic ribosomes initiate translation exclusively at the 5′ proximal AUG codon is abrogated only under rare conditions (see, e.g., Kozak PNAS 92(7): 2662-2666, (1995) and Kozak Nucl. Acids Res. 15(20): 8125-8148 (1987)).

In a further aspect, the invention is a pharmaceutical composition containing X-DING-CD4 peptide or its derivatives for preventing or treating a pathological condition in a mammal.

The term “mammal” refers to any organism classified as a mammal, including mice, rats, rabbits, dogs, cats, cows, horses and humans. In one embodiment of the invention, the mammal is a mouse. In another embodiment of the invention, the mammal is a human.

In some embodiments, the pathological condition is a viral infection and the peptides in this invention can prevent the viral infection. The preventative effect of the X-DING-CD4 peptide may be achieved, for example, by blocking the replication of virus inside cells and therefore preventing the virus from spreading over to other cells.

In some embodiments, the pathological condition is a viral infection and the peptides in this invention can treat the viral infection. The treatment effect of the X-DING-CD4 peptide and its derivatives may be achieved, for example, also by blocking the replication of virus inside cells and therefore preventing the virus from spreading over to other cells.

The viral infection can be a retroviral infection where retrovirus infects cells in a human body. For example, the retrovirus HIV-1 infects blood cells and can cause AIDS. In the instance of HIV-1 infection, X-DING-CD4 peptide and its derivatives may block the transcription of HIV-1 LTR through inhibition of NF-κB/DNA binding. X-DING-CD4 peptide and its derivatives may interact with p50NFκB after p50NFκB enters to nucleus but prior to its binding to DNA thus obstructing the formation of NFκB-DNA complex which is required for the initiation of LTR transcription, thereby blocking the NFκB-mediated biological pathway. The HIV-1 LTR is a common point for regulation by many cellular and viral proteins and these factors are attractive targets for antiviral treatment. To our knowledge, no antiviral therapy that targets HIV-1 LTR transcription is currently available. X-DING-CD4 peptide and its derivatives may block the LTR transcription, inhibit the replication of HIV-1, and thereby prevent or treat HIV-1 infection.

In other embodiments, the pathological condition is inflammation. X-DING-CD4 peptide and its derivatives may impede NF-κB-DNA binding in macrophages induced by either HIV-1 or lipopolysaccharide (LPS) from several bacteria species, resulting in impaired tumor necrosis factor-α (TNF-α) responses to these organisms and thereby blocking the NFκB-mediated biological pathway. The NF-κB system is involved in the immediate signaling mechanisms of the innate immunity responses against infecting pathogens, including HIV-1 (Hatada et al., 2000). Activation of NF-κB-dependent transcription of cytokines and other mediators of inflammation exerts protective anti-microbial function. Transcription of several viruses such as Herpes Simplex Virus (HSV), Simian Virus 40 (SV40) and HIV-1 depends on NF-κB signaling (Pahl, 1999). The activation of NF-κB is a common response to cellular exposure to many pathogens. For example, human beings infected with HIV-1 have greatly elevated levels of the Interleukin-8 (IL-8) protein which is mediated by NF-κB. Monocyte-derived macrophages and vascular endothelial cells undergo a rapid activation upon LPS stimulation resulting in the expression of several chemokines, including the IL-8 protein.

In another aspect, the invention is a method of preventing or treating a pathological condition by administrating a pharmaceutically effective amount of a pharmaceutical composition containing X-DING-CD4 peptide or its derivatives in a mammal. The peptides of the present invention may be provided per se or as part of a pharmaceutical composition, where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Hereinafter, the phrases “pharmaceutically acceptable carrier” refers to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Alternately, one may administer the pharmaceutical composition in a local rather than systemic manner, for example, via injection of the pharmaceutical composition directly into a tissue region of a patient.

Pharmaceutical compositions of the present invention may be formulated by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients (X-DING-CD4 peptide or its derivative) effective to prevent, alleviate or ameliorate symptoms of a disorder (e.g., AIDS/HIV infection) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to brain or blood levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on, for example, the subject being treated, the severity of the affliction, the manner of administration, and the judgment of the prescribing physician.

In some embodiments, the pharmaceutically effective amount X-DING-CD4 peptide or its derivatives is from about 1 nM to about 200 nM in blood stream, in particular from about 8 nM to about 150 nM, preferentially 100 nM. The concentration is estimated based on cell culture experiments and may be adjusted in human testing.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

In yet another aspect, the present invention is a composition comprising a polynucleotide encoding the X-DING-CD4 peptide having an amino acid sequence set forth in SEQ ID NO: 25 or a derivative of the X-DING-CD4 peptide, e.g., the peptides having amino acid sequences set forth in SEQ ID NOS: 1-15, for a cell-based therapy or a method for applying the above composition in a cell-based therapy. The cell-based therapy can be a gene therapy or a stem cell-based therapy. In a gene therapy, the polynucleotide encoding X-DING-CD4^{pep} or its derivative may be transiently or permanently expressed in target cells providing antiviral or anti-inflammatory protection. These target cells include, but are not limited to, peripheral mononuclear cells (PBMCs) or endothelial cells. In a stem cell-based therapy, patient's stem cells may be isolated and transformed to express X-DING-CD4 peptide or its derivatives, and the transformed individual stem cells may then be injected back into the patient to exert a short-term or a long-term anti-viral or anti-inflammatory protection.

In one embodiment of the cell-based therapy, the X-DING-CD4 peptide or its derivatives of the invention may be employed in accordance with the present invention by expression of such X-DING-CD4 peptide or its derivatives in vivo. Thus, for example, a virus may be engineered with a polynucleotide (DNA or RNA) encoding the X-DING-CD4 peptide or its derivatives, and the engineered virus is then provided to a patient to be treated with the X-DING-CD4 peptide or its derivatives. Such methods are well known in the art. For example, recombinant adenoviruses may be engineered by procedures known in the art containing DNA encoding the X-DING-CD4 peptide or its derivatives of the present invention.

In another embodiment of the cell-based therapy, the X-DING-CD4 cDNA may be employed in accordance with the present invention by expression of such X-DING-CD4 peptide or its derivatives in vivo. Thus, for example, cells may be engineered with a polynucleotide (DNA or RNA) encoding the X-DING-CD4 peptide having an amino acid sequence set forth in SEQ ID NO: 25 or its derivatives, e.g., the peptides having amino acid sequences set forth in SEQ ID NOS: 1-15 ex vivo, and the engineered cells are then provided to a patient to be treated with the X-DING-CD4 peptide or its derivatives. Such methods are well known in the art. For example, cells may be engineered by procedures known in the art by use of a retroviral particle containing RNA encoding the X-DING-CD4 peptide or its derivatives of the present invention.

Both in vitro and in vivo cell-based therapy methodologies are conceived. Several methods for transferring potentially therapeutic genes to defined cell populations are known. See, e.g., Mulligan (1993) Science 260:926-931. These methods include: 1) Direct gene transfer. Se, e.g., Wolff et al. (1990) Science 247: 1465-1468; 2) Liposome-mediated DNA transfer. See, e.g., Caplen et al. (1995) Nature Med. 3:3% 46; Crystal (1995) Nature Med. 1:15-17; Gao and Huang (1991) Biochem. Biophys. Res. Comm. 179:280-285; 3) Retrovirus-mediated DNA transfer. See, e.g., Kay et al. (1993) Science 262:117-119; Anderson (1992) Science 256:808-813.4) DNA Virus-mediated DNA transfer. Such DNA viruses include adenoviruses (preferably Ad2 or Ad5 based vectors), herpes viruses (preferably herpes simplex virus based vectors), and parvoviruses (preferably “defective” or non-autonomous parvovirus based vectors, more preferably adeno-associated virus based vectors, most preferably AAV-2 based vectors). See, e.g., Ali et al. (1994) Gene Therapy 1:367-384; U.S. Pat. No. 4,797,368, incorporated herein by reference, and U.S. Pat. No. 5,139,941, incorporated herein by reference.

The choice of a particular vector system for transferring the gene of interest may depend on a variety of factors. One important factor is the nature of the target cell population. Although retroviral vectors have been extensively studied and used in a number of gene therapy applications, these vectors are generally unsuited for infecting non-dividing cells. In addition, retroviruses have the potential for oncogenicity. However, developments in the field of lentiviral vectors may circumvent some of these limitations. See Naldini et al. (1996) Science 272:263-267.

Retroviruses from which the retroviral plasmid vectors hereinabove mentioned may be derived include, but are not limited to, Moloney Murine Leukemia Virus, spleen necrosis virus, retroviruses such as Rous Sarcoma Virus, Harvey Sarcoma Virus, avian leukosis virus, gibbon ape leukemia virus, human immunodeficiency virus, adenovirus, Myeloproliferative Sarcoma Virus, and mammary tumor virus.

Adenoviruses have the advantage that they have a broad host range, can infect quiescent or terminally differentiated cells, such as neurons or hepatocytes, and appear essentially non-oncogenic. See, e.g., Ali et al. (1994), supra, p. 367. Adenoviruses do not appear to integrate into the host genome. Because they exist extrachromosomally, the risk of insertional mutagenesis is greatly reduced. Ali et al. (1994), supra, p. 373.

In a preferred embodiment, the DNA encoding the X-DING-CD4 peptide or its derivatives of this invention is used in cell or gene therapy for HIV infections or inflammation reactions. In some instances, the cell-based therapy with DNA encoding the X-DING-CD4 peptide or its derivatives of this invention is provided to a patient in need thereof, concurrent with, or immediately after diagnosis of HIV infections or inflammation reactions. In other instances, the cell-based therapy with DNA encoding the X-DING-CD4 peptide or its derivatives of this invention is provided to a patient in need thereof to prevent the occurrence of HIV infections or inflammation reactions.

The skilled artisan will appreciate that any suitable cell-based therapy vector containing DNA encoding the X-DING-CD4 peptide or its derivatives of the invention may be used in accordance with this embodiment. The techniques for constructing such a vector are known. See, e.g., Anderson, W. F. (1998) Nature 392:25-30; Verma I. M. and Somia, N. (1998) Nature 389:239-242. Introduction of the antibody DNA-containing vector to the target site may be accomplished using known techniques.

The cell-based therapy vector may include one or more promoters. Suitable promoters which may be employed include, but are not limited to, the retroviral LTR; the SV40 promoter; and the human cytomegalovirus (CMV) promoter described in Miller et al. (1989) Biotechniques 7(9):980-990, or any other promoter (e.g., cellular promoters such as eukaryotic cellular promoters including, but not limited to, the histone, polymerase III, and β-actin promoters). Other viral promoters which may be employed include, but are not limited to, adenovirus promoters, thymidine kinase (TK) promoters, and B19 parvovirus promoters. The selection of a suitable promoter will be apparent to those skilled in the art from the teachings contained herein.

The nucleic acid sequence encoding the X-DING-CD4 peptide or its derivatives of the present invention is under the control of a suitable promoter. Suitable promoters which may be employed include, but are not limited to, adenoviral promoters, such as the adenoviral major late promoter; or heterologous promoters, such as the cytomegalovirus (CMV) promoter; the respiratory syncytial virus (RSV) promoter, inducible promoters, such as the MMT promoter, the metallothionein promoter; heat shock promoters; the albumin promoter; the ApoAI promoter; human globin promoters; viral thymidine kinase promoters, such as the Herpes Simplex thymidine kinase promoter; the β-actin promoter, and human growth hormone promoter.

The retroviral plasmid vector is employed to transduce packaging cell lines to form producer cell lines. Examples of packaging cells which maybe transfected include, but are not limited to, the PE501, PA317, .psi.-2, .psi.-AM, PA12, T19-14×; VT-19-17-H2, .psi.CRE, .psi.CRIP, GP+#-86, GP+envAm12, and DAN cell lines as described in Miller (1990) Human Gene Therapy 1:5-14, which is incorporated herein by reference in its entirety. The vector may transduce the packaging cells through any means known in the art. Such means include, but are not limited to, electroporation, the use of liposomes, and CaPO₄ precipitation. In one alternative, the retroviral plasmid vector may be encapsulated into a liposome, or coupled to a lipid, and then administered to a host. The producer cell line generates infectious retroviral vector particles which include the nucleic acid sequence(s) encoding the polypeptides. Such retroviral vector particles then may be employed, to transduce eukaryotic cells, either in vitro or in vivo. The transduced eukaryotic cells will express the nucleic acid sequence(s) encoding the polypeptide. Eukaryotic cells which may be transduced include, but are not limited to, embryonic stem cells, embryonic carcinoma cells, as well as hematopoietic stem cells, hepatocytes, fibroblasts, myoblasts, keratinocytes, endothelial cells, and bronchial epithelial cells.

In another embodiment, the patient's cells are treated ex vivo to induce the dormant chromosomal genes to produce the X-DING-CD4 peptide or its derivatives after reintroduction to the patient. In this embodiment, a promoter or other exogenous regulatory sequence capable of activating the X-DING-CD4 gene, or its fragment or homologue, into the chromosomal DNA of the patients' cells ex vivo, culturing and selecting for active protein-producing cells, and then reintroducing the activated cells into the patient with the intent that they then become fully established. The activated cells then manufacture the X-DING-CD4 peptide or its derivatives for some significant amount of time, perhaps for as long as the life of the patient. U.S. Pat. Nos. 5,641,670 and 5,733,761 disclose in detail this concept, and are hereby incorporated by reference in their entirety.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

It should be understood that this invention is not limited to the particular methodologies, protocols and reagents, described herein, which may vary. The terminology used herein is for the purpose of describing particular embodiments only, and is not intended to limit the scope of the present invention.

Examples of the disclosed subject matter are set forth below. Other features, objects, and advantages of the disclosed subject matter will be apparent from the detailed description, figures, examples and claims. Methods and materials substantially similar or equivalent to those described herein can be used in the practice or testing of the presently disclosed subject matter. Exemplary methods and materials are now described as follows.

### Example 1

### Cloning of X-DING-CD4 cDNA and Generation of X-DING-CD4 Peptide by Recombinant Protein Expression

The complete genomic sequence for human X-DING-CD4 gene has never been defined and the chromosome localization of human X-DING-CD4 is not known. It is likely that based on its very high GC content, this gene may be localized in a centromere, which has not been completed in any human genome sequencing project. The X-DING-CD4 gene is characterized with high GC content, high level of secondary structures and possibly rapid renaturation into a highly folded form. Conventional or standard amplification methods by other or us have failed to amplify the X-DING-CD4 gene.

We cloned the partial cDNA encoding X-DING-CD4 peptide using a forced linearization of DNA template by co-precipitation of denatured cDNA with a neutral nucleic acid carrier. Specifically, total RNA was isolated from X-DING-CD4 positive cells using the Trizol reagent (Invitrogen) according to manufacturer's protocol. Two μg of total RNA sample pre-treated with DNAse I was reverse transcribed using random hexamers and gene specific primer of X-DING-CD4 gene (RD1-5′-TCGTTCAGCTTGGTCATCACGCCTTGGCTGGTCACGGC-3′, SEQ ID NO: 17) and Transcriptor First Strand cDNA synthesis kit (Roche) according to manufacturer's protocol with the exception that the annealing temperature was set at 65° C. The cDNA was amplified with AccuPrime GC-Rich DNA polymerase (Invitrogen) using two pairs of gene specific primers appended with BamHI and HindIII restriction sites as follows: forward 5′-GCG CGC GGA TCC GCC ATG GCA GAC ATC AAC GGC GGC GGC-3′ (SEQ ID NO: 18) and reverse 5′-CGC GGC AAG CTT TCC TGG CTG AAG ATC AGG TTG GT-3′ (SEQ ID NO: 19). The PCR products were purified with a gel extraction kit (Qiagen, Germany) and cloned into pCR™2.1-TOPO vector (Invitrogen), and verified by sequencing using M13 primers. The cloned cDNA was confirmed to have the nucleotides set forth in SEQ ID No: 26.

Following the same procedure except that the reverse primer (SEQ ID NO: 19) was replaced with the new reverse primer 5′-CGC GGC AAG CTT TCG TTC AGC TTG GTC ATC ACG CCT TGG CTG G-3′ (SEQ ID NO: 28), we cloned the partial X-DING-CD4 cDNA having the nucleotides as set forth SEQ ID NO: 16, which encodes X-DING-CD4^{pep} having an amino acid sequence as set forth in SEQ ID NO: 1. We then subcloned the partial X-DING-CD4 cDNA (SEQ ID NO: 16) thus obtained into a bacterial expression vector. Specifically, the X-Ding-CD4/TOPO clone was digested with BamHI and HindIII enzymes and ligated into pET-28a vector (Novagen, Madison, USA). The ligated product was transformed into E. coli BL21 (DE3pLysS) cells. The positive clones were designated pET28a/X-DING-CD4. Transformed BL-21 cells were amplified by growing on LB broth medium (Gibco) supplemented with 50 μg/mlkanamycin (Sigma). The transformed cells were grown to log phase (OD600=0.6 to 1.0) and transcription of recombinant protein was induced by IPTG at concentration of 1 mM at 37° C.

The recombinant X-DING-CD4^{pep} was purified using QIAexpress Ni-NTA Fast Start kit (Qiagen, Germany) under native conditions according to the manufacturer's protocol. Briefly, the induced E. coli (BL21) culture was collected and re-suspended in 10 ml of Lysis buffer containing Lysozyme and Benzonase Nuclease. The cells were incubated on ice for 30 min and Mixed 2-3 times by gently swirling the cell suspension. The lysate was centrifuged at 14,000×g for 30 min at 4° C. to pellet the cellular debris and collect the cell lysate supernatant. The supernatant contained the soluble fraction of the recombinant protein.

The Ni-NTA resin was gently re-suspended in a Fast Start Column by inverting it several times. The cell lysate supernatant was applied to the column and the column was washed twice with 4 ml of native Wash Buffer. The bound 6×His-tagged protein was eluted with two 1 ml aliquots of Native Elution Buffer. Each elution fraction was collected in a separate tube and aliquoted for storage at −20° C. The purity and specificity of eluents was established by SDS-PAGE using Coomassie blue and Western Blot using Monoclonal X-DING-CD4 antibody (mouse monoclonal to X-DING-CD4).

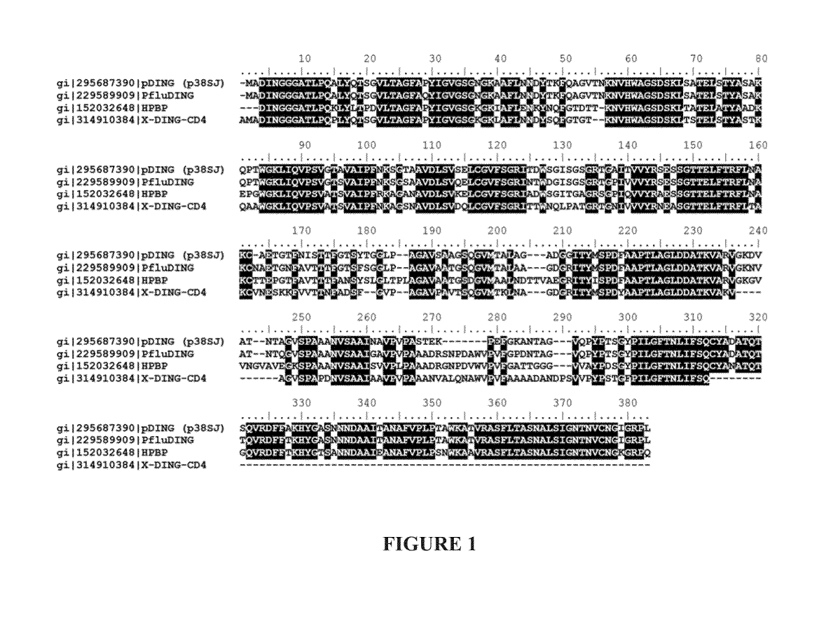

X-DING-CD4 peptide has distinct features that are not present in other DING family proteins. A sequence comparison of X-DING-CD4 peptide (SEQ ID NO: 25) with PfluDING from Pseudomonas fluorescens, pDING from Hypericum perforatum, and human phosphate binding protein (HPBP) from human plasma (Chemier et al., 2011) is shown in FIG. 1. X-DING-CD4 protein comprises of two forms distinguished by methylation of glutamic acid in amino acid position 69. Methyl esterification of glutamic acid was first reported as a novel posttranslational modification distinguishing diverse isoforms of proliferating cell nuclear antigen (PCNA). The percentage identity between X-DING-CD4^{pep} and similar amino acid regions in HPBP and p27SJ is 65% and 67%, respectively. The isolated X-DING-CD4 cDNA (SEQ ID NO: 26) encodes the X-DING-CD4 peptide (SEQ ID No: 25). Four myristoylation sites encompass 19-36, 61-78, 94-111 and 316-363 nucleotides. Specific globular protein binding domains within X-DING-CD4 peptide amino acid sequence includes:

(1) FxDxF (FADSF aa 72-176)—motif responsible for the binding of accessory endocytic proteins to the appendage of α-subunit of adaptor protein complex AP-2;

(2) ESKKF (aa 162-166) phosphopeptide motif which interacts directly with the BRCT (carboxy-terminal domain) of the BRCA1;

(3) KLTSTEL (aa 64-70) responsible for phosphothreonine binding to FHA domains; KGKLAFL (aa 36-42) and GTKNVHWA (aa 52-59) binding to protein phosphatase 1 catalytic subunit (PP1c) docking proteins for Dephosphorylation;

(4) APYIGVG (aa 27-33) calcineurin substrate docking site;

(5) AFLNNDYS (aa 40-47) phosphorylation independent motif binding Dab-like PTB;

(6) multiple GSK3 phosphorylation recognition sites (aa—16-23; 44-51; 60-67; 64-71; 65-72; 68-75; 69-76; 73-80; 86-93; 115-122; 145-152; 149-156);

(7) VNESKK (aa 160-165) generic motif for N-glycosylation;

(8) NDYSQFG (aa44-50) and PVTSQGV (aa 184-190) motif recognized by PIKK kinases

(9) YIGV (aa 29-32) and YSQF (aa 46-49) motifs are tyrosine based sorting signals responsible for the interaction with mu subunit of AP (adaptor protein complex).

### Example 2

### X-DING-CD4 Peptide Blocks HIV-1 LTR Promoter Transcription and HIV-1 Replication

This example shows that X-DING-CD4 peptide could block the transcription of HIV-1 LTR promoter. We applied the cell based Rapid Suppression Assay (RSA) for testing X-DING-CD4-induced inhibition of HIV-1 LTR promoter (Lesner et al., 2004; Lesner et al., 2005). Briefly, 1 G5 cells (obtained through the NIH AIDS Research and Reagent Program, Division of AIDS, NIAID, NIH) that are stably transfected with an inducible luciferase gene driven by HIV-1 LTR (Aguilar-Cordova et al., 1994) were washed in PBS, resuspended in hybridoma medium to concentration of 5×10⁶cells/ml. For purpose of generating control titration curves, 100 μl aliquots of 1G5 cells were mixed with 100 μl cell culture medium either supplemented with various amount of rX-DING-CD4^{pep} (SEQ ID No: 1) or no rX-DING-CD4^{pep}, and incubated for three hours at 37° C. Subsequently, all cells were induced with phorbol 12-myristate 13-acetate (PMA) at a concentration of 5 ng/ml. Two control tubes containing 1G5 cells without rX-DING-CD4^{pep} treatment were resuspended in hybridoma medium with or without PMA. Three hours later cells were collected by centrifugation and lysed in the same tubes using Reporter Lysis buffer (Promega). The expression levels of luciferase protein in the lysates were determined according to the manufacturer's protocol. The X-DING-CD4^{pep} inhibition values were established based on the formula: [(LUC₁-LUC₀)×100/Z]−100; where LUC₁ is the value obtained from cells induced by PMA and treated with a specific dilution of X-DING-CD4^{pep}; and LUC₀ is the basal value obtained from the uninduced and untreated cells; Z is the absolute luciferase induction by PMA calculated as Z=LUCₘₐₓ-LUC_{O3} where LUCₘₐₓ is the value of 100% luciferase expression in PMA induced X-DING-CD4^{pep} untreated cells.

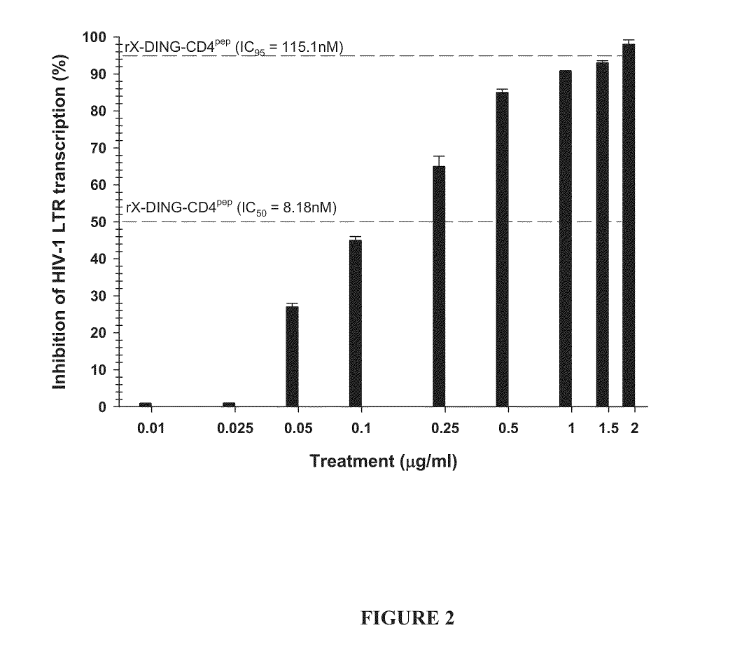

As shown in FIG. 2, rX-DING-CD4^{pep} could inhibit PMA-induced transcription of HIV-1 LTR promoter. As the amount of rX-DING-CD4^{pep} used for cell treatment increases (the final concentration at 0.01 m/ml, 0.025 μg/ml, 0.05 μg/ml, 0.1 μg/ml, 0.25 μg/ml, 0.5 μg/ml, 1.0 μg/ml, 1.5 μg/ml, and 2.0 μg/ml), the percentage of transcription being inhibited increases. The inhibitory dose blocking 95% or 50% transcription events (IC₉₅ or IC₅₀) for rX-DING-CD4^{pep} was about 115.1 nM (1.52 μg/ml) and about 8.18 nM (0.1 m/ml), respectively. No LTR inhibition was observed in X-DING-CD4^{pep} untreated cells.

In addition, rX-DING-CD4 peptide could block HIV-1 replication. The antiviral activity of rX-DING-CD4^{pep} was tested by MAGI assay. MAGI assay was described in prior publications, e.g., in (Chackeri an et al 1997) Briefly: MAGI-CCR-5 cells were seeded 24 hour prior to assay in a 96-well plate (Costar) at 6.2×10³ cells per well in DMEM supplemented with 10% fetal bovine serum, antibiotics and glutamate. Subsequently, cells were exposed to rX-DING-CD4^{pep} treatment in the presence of DMEM supplemented with 5% fetal bovine serum, antibiotics, glutamate and 20 m/ml DEAE dextran. Twenty four hours later cells were infected with 0.1 pg/cell NL4-3 HIV-1 isolate (Adachi et al., 1986). Replication of virus was evaluated forty eight hours later after fixation of cells with 1% formaldehyde and 0.2% glutaraldehyde in PBS. Expression of β-galactosidase was visualized by 50 min exposure to X-GAL (5-bromo-4-chloro-3-indolyl-β-D-galactopyranoside), 0.4 mg/ml 2 mM MgCl₂, 4 mM potassium ferricyanide at 37° C. After enumeration of the infected (blue) cells, all cells were lysed and subjected to the protein assay (Bio-Rad). All data were adjusted by total protein concentration and anti-viral activity was calculated as percent inhibition. The infected untreated and uninfected cells served as (+) and (−) controls to calculate 100% virus infectivity. The inhibition values for HIV-1 replication were calculated based on the formula [(R₁−R₀)×100/Z]-100; where R₁ is the value obtained from cells infected by HIV-1 and treated with specific dilution of X-DING-CD4^{pep} and R⁰ is the basal value obtained from the uninfected and untreated cells; Z is the absolute HIV-1 replication value calculated as Z═Rₘₐₓ−R₀, where Rₘₐₓ is the value representing 100% of HIV-1 replication in X-DING-CD4^{pep} untreated cells.

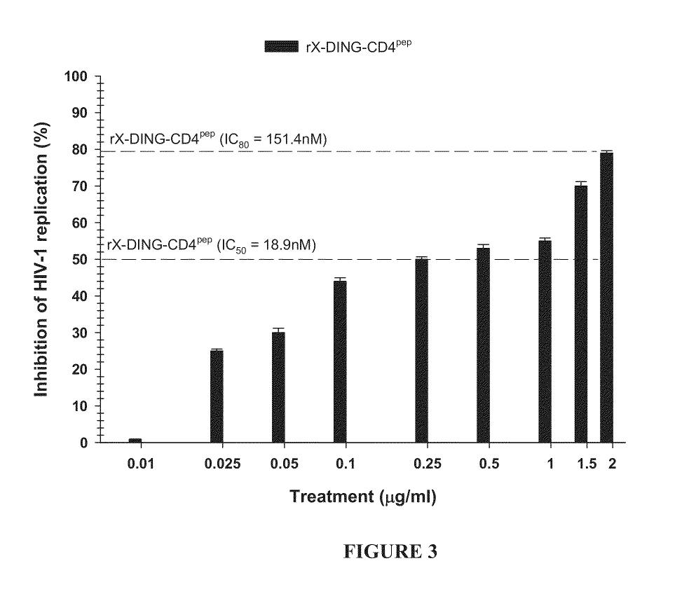

As shown in FIG. 3, rX-DING-CD4^{pep} blocked replication of HIV-1. The inhibitory dose blocking 80% or 50% transcription events (IC₈₀ or IC₅₀) for rX-DING-CD4^{pep} was 151.1 nM (2 m/ml) and 18.9 nM (0.5 m/ml), respectively. No HIV-1 inhibition was observed in X-DING-CD4^{pep} untreated cells.

The rX-DING-CD4^{pep} retains anti-HIV-1 biological activity of the whole native X-DING-CD4 protein. Native X-DING-CD4 refers to extracellular X-DING-CD4 form of about 40 kDa that is secreted by HIV resistant cells. The cellular uptake of rX-DING-CD4^{pep} is receptor independent which increases the success rate of its therapeutic delivery to target cells. This particular receptor-independent transmembrane permeability of native X-DING-CD4 and X-DING-CD4′ was confirmed experimentally in cells exposed to 125 ng/ml trypsin. Therapeutic potential of rX-DING-CD4^{pep} to block transcription of HIV-1 promoter is very high: the inhibition of 90% HIV-1 LTR transcription can be achieved at 115.1 nM concentration. Inhibition of 80% HIV-1 replication can be achieved in 151.4 nM concentration.

### Example 3

### Administration of rX-DING-CD4Pep does not Cause Acute Toxicity in Animal Tests

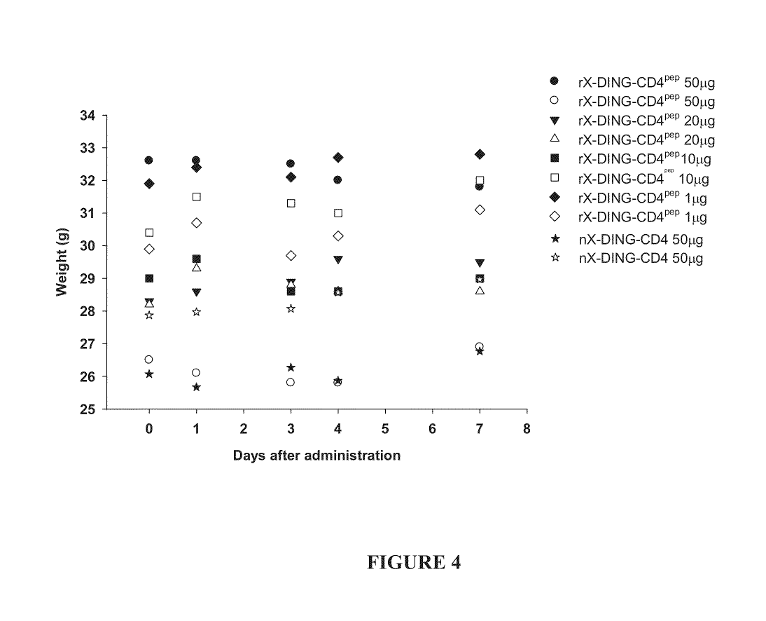

X-DING-CD4 peptide displays low toxicity in animal tests and therefore may be a pharmaceutical composition in treating animals and human beings. In the animal tests, eight-week old wild-type mice (B6/SJL) with both genders in each test group were injected intraperitoneally with 1-50 μg of rX-DING-CD4^{pep} or 50 μg nX-DING-CD4. The nX-DING-CD4 refers to the native form of X-DING-CD4 protein that was purified through IXC chromatography from the extracellular compartment of HIV-1 resistant T-cells. The rX-DING-CD4^{pep} refers to therapeutic X-DING-CD4 peptide that was expressed in E. coli and purified through Ni-affinity column. Toxicity was assessed by obvious symptoms including diarrhea, ruffled fur, abnormal gait, or loss of more than 10% weight. Animals were weighted before injection and then observed every hour for the first 10 hours after injection; then animals were weighted on 1, 3, 4 and 7 days after X-DING-CD4 administration. Neither of rX-DING-CD4^{pep} treatment nor nX-DING-CD4 treatment caused any obvious symptoms including diarrhea, ruffled fur, and abnormal gait. Further, as shown in FIG. 4, none of the treated animals displayed more than 10% variation in weight. In its highest administered dose (50 μg—corresponding to 2 μg/G) we exceeded therapeutic dose blocking 95% of HIV promoter activity (by RSA assays) and 90% virus replication (by MAGI assays) (See FIGS. 2 & 3 in Example 2).

### Example 4

### Anti-HIV-1 Activity of X-DING-CD4 Peptide Derivatives

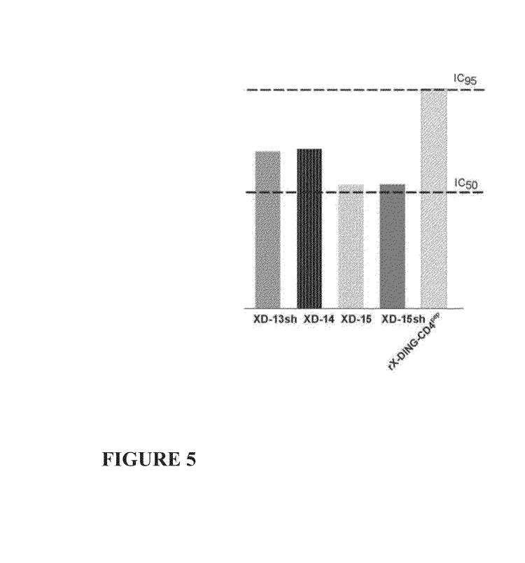

One aspect of the present invention is related to X-DING-CD4 peptide derivatives. Here we obtained 15 derivatives SED ID NOS: 1-15 by recombination or in vitro synthesis. We tested the biological activity of some these derivatives by rapid suppression assay (RSA). In XD-4M, the third amino acid glycine in the native X-DING-CD4 peptide was substituted with the polar amino acid glutamine. In XD-7, the two amino acids, cysteine and valine, replaced the three amino acids, glycine, serine and aspartic acid, in the native X-DING-CD4 peptide. In XD-12 and XD-12sh, the 69^{th} amino acid glutamic acid in the native X-DING-CD4 peptide was methylated. In XD-9, the 69^{th} amino acid glutamic acid in the native X-DING-CD4 peptide was substituted with the amino acid glutamine. The RSA procedure was similar to that described in Example 2. The derivative peptide sequences and their inhibitory concentration (IC) values are listed in Table 1. The inhibitory values are plotted in FIG. 5.

Some of these derivatives may also have therapeutic activities. For example, at a concentration of 0.337 μM, 50% of LTR promoter transcription was inhibited XD-12. Therefore, some derivatives can also be pharmaceutical compositions. The procedure of assaying inhibitory activities of derivative peptides as disclosed herein can be followed, as a matter of routine experimentations, to identify other derivative peptides with therapeutic activities.

TABLE 1
HIV-1 LTR promoter inhibition by X-DING-CD4 peptide and its derivatives.
MW refers to molecular weight.

| SEQ | | | | |
|---|---|---|---|---|
| ID | Peptide | | | |
| NO: | name/MW | Amino acid sequence | IC₅₀ | IC₉₅ |
| 1 | rX-DING-CD4 | MADINGGGATLPQPLYQTSGVLTAGFAPYIGV | 8.2 nM | 0.116 μM |
| | MW = 13048 g/mol | GSGKGKLAFLNNDYSQFGTGTKNVHWAGSDS | (0.107 ug/ml) | (1.514 μg/ml) |
| | | KLTSTELSTYASTKQAAWGKLIQVPSVATSVAI | | |
| | | PFNKAGSNAVDLSVDQLCGVFSGRITTWNQ | | |
| 2 | XD-2 | GTGTKNVHWAGSDSKLTATELSTYATDK | ND | ND |
| | MW = 2940 g/mol | | | |
| 3 | XD-3 | LAFLNNDYSQEGTGTKNVHWAGSDSK | ND | ND |
| | MW = 2840 g/mol | | | |
| 4 | XD-4M | DINGGQTLPQK | 57 nM | ND |
| | MW = 1170 g/mol | | (66.9 ng/ml) | |
| 5 | XD-5 | GTKNVHWAGSDSKLTSTELSTYC | 35.8 nM | ND |
| | MW = 2486 g/mol | | (88.7 ng/ml) | |
| 6 | XD-7 | GTKNVHWACVSKLTSTELSTY | 29.8 nM | ND |
| | MW = 2325 g/mol | | (69.3 ng/ml) | |
| 7 | XD-12 | GVSNKNVHWAGSDSKLTATE(OMe)LSTY | 33.7 nM | ND |
| | MW = 2556 g/mol | | (86.2 ng/ml) | |
| 8 | XD-12sh | YTSLE(OMe) | 121 nM | ND |
| | MW = 612 g/mol | | (74 ng/ml) | |
| 9 | XD-13 | GVSNKNVHWAGSDSKLTATQLSTY | 30.2 nM | ND |
| | MW = 2565 g/mol | | (77.5 ng/ml) | |
| 10 | XD-13sh | QLSTY | 121 nM | ND |
| | MW = 611 g/mol | | (74 ng/ml) | |
| 11 | XD-14 | GVSNKNVHWAGSDSKLTAT | 36.8 nM | ND |
| | MW = 1972 g/mol | | (72.5 ng/ml) | |
| 12 | XD-15 | GVSNKNVHWAGSDSKLTATLLSTY | 36.7 nM | ND |
| | MW = 2550 g/mol | | (93.5 ng/ml) | |
| 13 | XD-15sh | LLSTY | 156.9 nM | ND |
| | MW = 596 g/mol | | (93.5 ng/ml) | |
| 14 | XD-1 | DINGGGATLPQK | 51.2 nM | ND |
| | MW = 1170 g/mol | | (60 ng/ml) | |
| 15 | XD-4 | KNVHWAGSDSKLTSTELSTY | 31 nM | ND |
| | MW = 2224 g/mol | | (70 ng/ml) | |

### Example 5

### Anti-HIV-1 activity of rX-DING-CD4^{pep} in a HIV mouse model

This example shows that rX-DING-CD4^{pep} demonstrates therapeutic anti-HIV activity in a mouse model of HIV infection. The mouse model of HIV infection is well-known by persons skilled in the art and is widely used to study therapeutic activities of various pharmaceutical agents. It was generated by using chimeric HIV (EcoHIV), which is a modified HIV that can infect mouse T cells and macrophages and can establish persistent infection of immunocompetent mice after intravenous or intraperitoneal exposure (Potash et al., 2005). The EcoHIV infection mouse model reproduces many characteristics of HIV-1 infection in humans such as virus targeting to lymphocytes and macrophages, induction of antiviral immune responses, neuroinvasiveness, and elevation of expression of inflammatory and antiviral factors in the brain (Potash et al., 2005). This model has been used to study the efficacy of anti-HIV vaccines (Roshorm et al., 2009; Saini et al., 2007), the mechanism of HIV integrase inhibitory peptide (Hayouka et al., 2010), and the efficacy of nucleoside reverse transcriptase inhibitors NRTI and ddC (Hadas et al., 2007).

In this example, we started with three groups of C57BL mice (groups I, II and III), each group having three mice. We injected into peritoneum with 20 μg of rX-DING-CD4^{pep}, 0.9% saline, and heat inactivated (HI-rX-DING-CD4^{pep}) protein in groups I, II, and III, respectively. Three hours later all animals were infected by peritoneum injection with 0.5×10⁶ pg ecotropic HIV-1 EcoNDK according to the procedure described in (Potash et al., 2005). Three hours after the virus injections, we repeated peritoneum injections with 20 μg of rX-DING-CD4^{pep}, 0.9% saline, and heat inactivated HI-rX-DING-CD4^{pep} protein in groups I, II, and III, respectively. After 48 hours all animals were sacrificed to harvest spleen and peritoneal macrophages (pMQ) to evaluate the EcoHIV-1 virus load. The amount of EcoHIV-1 virus load was assessed by determining the messenger RNA (mRNA) levels of HIV-Tat and Vif genes by quantitative RT-PCR (qRT-PCR). The amount of HIV-1 Gag DNA in the each sample, which was also determined by qRT-PCR, was used as a quantitative control for the HIV-Tat and Vif mRNA levels. The detailed procedure, the primers and the reaction conditions were described in (Potash et al., 2005) and hereby incorporated by reference in its entirety.

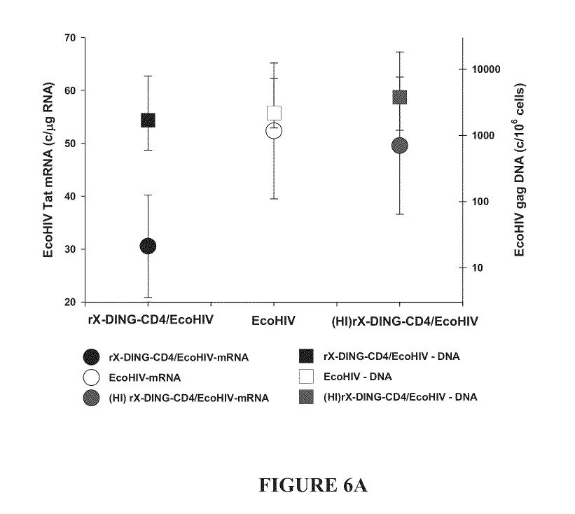

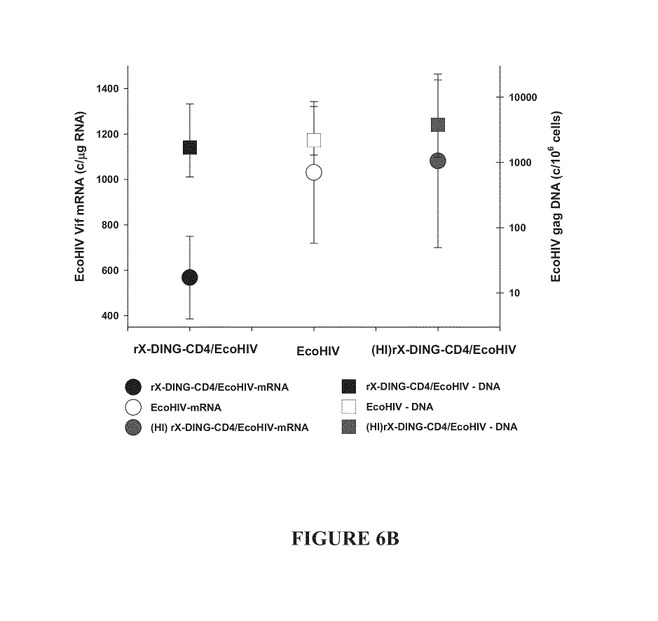

As shown in FIGS. 6A and 6B, 75% transcription of EcoHIV was successfully blocked by only two doses of rX-DING-CD4^{pep} peptide two days after infection. In group I, decreased expression of EcoHIV Tat mRNA and vif mRNA was observed in comparison to group II without protein treatment or group III treated with heat inactivated rX-DING-CD4^{pep}.

The therapeutic activity of rX-DING-CD4^{pep} in the HIV mouse model suggests that similar effects in blocking HIV replication are expected in human individuals infected with HIV. Together with the results shown in Example 4, other derivatives of X-DING-CD4 peptide are also conceived here to have therapeutic effects in blocking HIV-1 replication in humans.

### Example 6

### Anti-HIV-1 Activities of X-DING-CD4

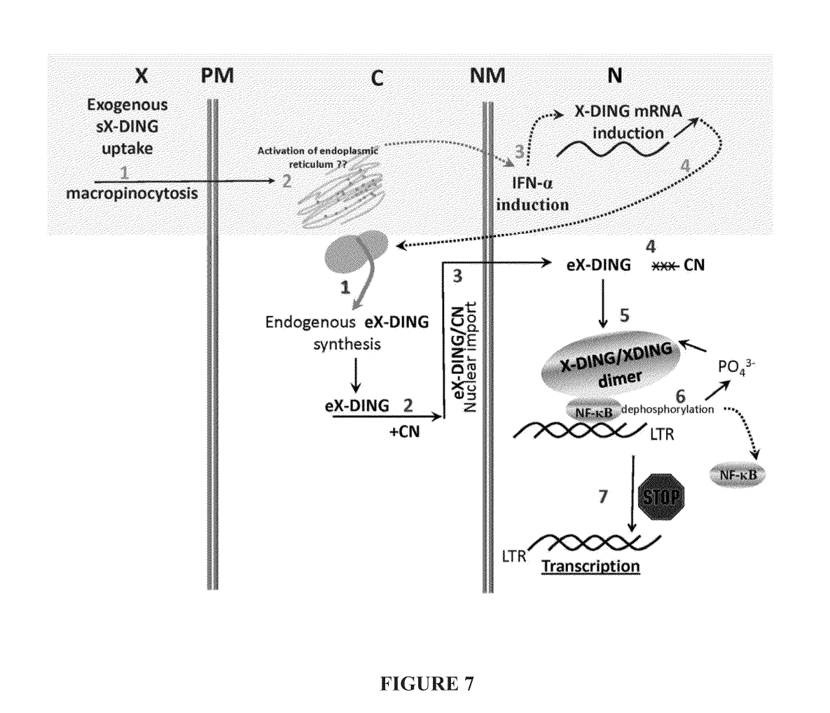

We conceived the mechanism of how X-DING-CD4 peptide works to suppress HIV-1 transcription inside a cell. See FIG. 7 and the below text for details.

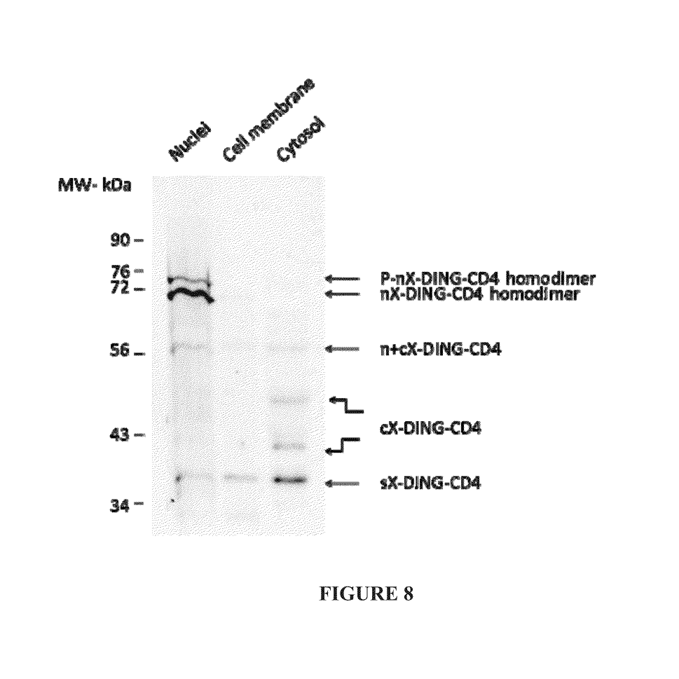

As shown in FIG. 8, different forms of X-DING-CD4 exist in different cellular compartments: nuclei, cell membrane, and cytosol. In this example, the subcellular compartments of X-DING-CD4(+) cells were isolated as described before (Lesner et al., 2004) and intracellular X-DING-CD4 protein forms were resolved through SDS PAGE followed by blotting with monoclonal antibody to X-DING-CD4 (mouse monoclonal to X-DING-CD4) in accordance with regular procedures known in the art. As shown in the lane “Nuclei”, two forms of nuclear X-DING-CD4 homodimers exited. These two homodimers differ by the presence or absence of phosphate polyatomic ion (PO₄³). The form without phosphate polyatomic ion is designated as nX-DING-CD4. The form with phosphate polyatomic ion is designated as P-nX-DING-CD4.

As shown in the lane “cell membrane”, sX-DING-CD4 exists in cell membrane, indicating it being transported from cytosol to extracellular space. As shown in the lane “cytosol”, three forms of X-DING-CD4 exist: cX-DING-CD4 referring to the protein form present only in cytosol; n+cX-DING-CD4 referring to protein forms present in both nuclear and cytosolic fractions; and sX-DING-CD4 referring to processed soluble form X-DING-CD4 of about 39 kDa, which may be exported through cell membrane to extracellular space and is found preferably in the extracellular compartment.

The exogenous X-DING-CD4 (sX-DING-CD4) is transported to cell through macropinocytosis, possibly through its association with HDL and LDL. We expect that alongside the cholesterol cellular trafficking the sX-DING-CD4 is released to endoplasmic reticulum (ER), resulting in activation of ER and induction of IFN-α. IFN-α induces expression of endogenous X-DING (eX-DING-CD4) mRNA. The nuclear import of eX-DING-CD4 is enabled by transient complex with calcineurin (CN) mediated by motif PxIxVG present on eX-DING-CD4 and a docking groove present on CN. The eX-DING-CD4/CN complex dissociates in the nucleus and eX-DING-CD4 forms homodimers. The eX-DING-CD4 homodimer binds NF-κB/LTR complex through 9 amino acid transactivation domain (9AATAD) present on eX-DING-CD4 and dephosphorylates NF-κB leading to its dissociation from the LTR. The mechanism of sX-DING-CD4 can be reproduced by X-DING-CD4^{pep} and several N′-terminal synthetic peptides. Determination of globular domains and signal peptide domains was performed using Eukaryotic Linear Motif resource through the ExPASy Bioinformatics Resource Portal from Swiss Institute of Bioinformatics.

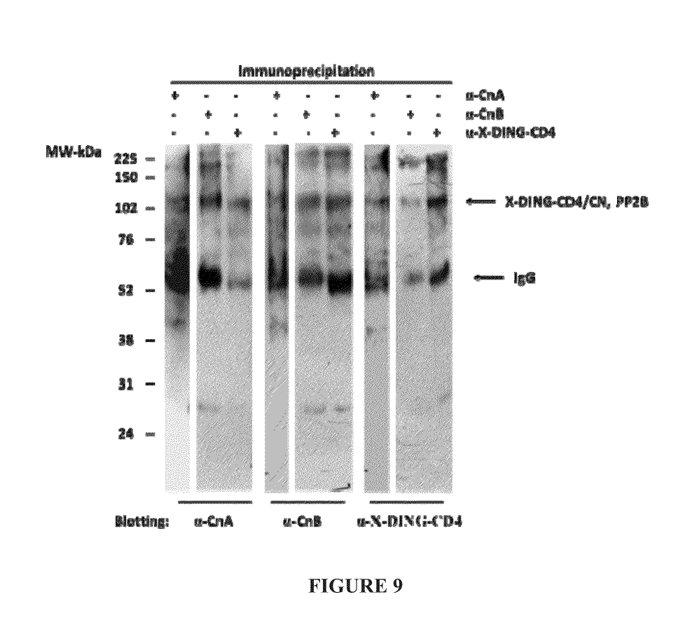

The immunoprecipitation (IP) of endogenous X-DING-CD4 was performed upon X-DING-CD4(+) cell lysates with antibodies specific to calcineurin A and B (CnA, CnB) and X-DING-CD4. Precipitated proteins were resolved through SDS PAGE and detected with antibodies reciprocal to those used in the IP. As shown in FIG. 9, X-DING-CD4/CN complex can be detected in the precipitates from each of CnA, CnB and X-DING-CD4.

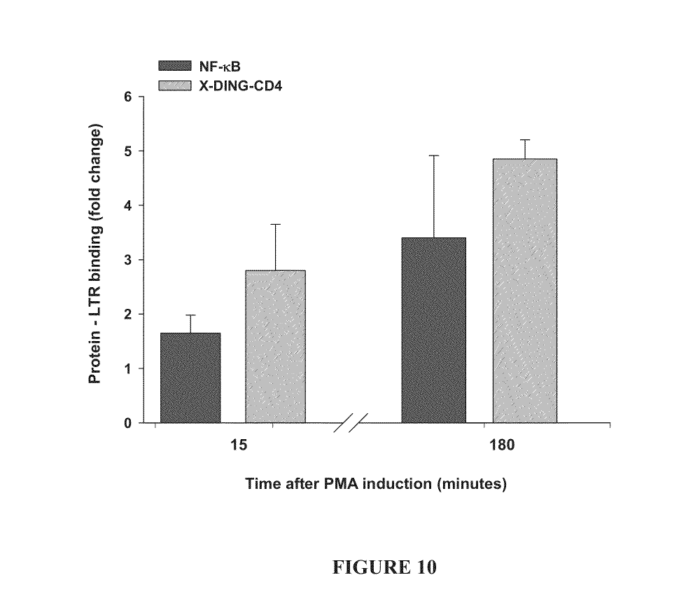

In the nucleus, the endogenous X-DING-CD4, hereby referred to as eX-DING for reasons of simplicity, dissociates from CN (step 4) and forms homodimers (step 5 and FIG. 8, see the lane “nuclei”). The eX-DING homodimer binds the NF-κB/LTR complex (FIG. 10) through the 9aa transactivation domain (TAD) present on X-DING-CD4. Determination of 9aa TAD was performed using 9aa TAD Prediction Tool through the ExPASy Bioinformatics Resource Portal from Swiss Institute of Bioinformatics. Based on this analysis the 9aa TAD motif on X-DING-CD4 is located at amino acid position 149-157 and was specified as a perfect match for sequence (ELFTRFLTA), SEQ ID NO: 27.

Association of X-DING-CD4 with NF-κB/LTR complex was determined by qRT-PCR based chromatin precipitation (ChIP) assay that was developed in our laboratory and described in the (Ivanova et al., 2012), which description is hereby incorporated by reference in its entirety.

Briefly, 1G5 cells were cultured for 3 hrs in the presence of 100 ng native X-DING-CD4 protein. Subsequently, cells were washed in PBS and medium was replaced by RPMI supplemented with PMA. At designated time points all proteins were cross-linked with 1%/vol HCHO for 10 min. at 25° C. followed by 5 min. incubation in 0.125M glycine. Cells were washed in PBS and resuspended in ice-cold lysis buffer (50 mM Tris pH 8.0, 0.2 mM EDTA, 0.1%/vol NP40, 10%/vol glycerol and standard cocktail of protease inhibitors: Aprotinin 1 μg/ml, Leupeptin 1 μg/ml and AEBSF 5 μg/ml (all from Sigma)); 10 min. later nuclei were collected by centrifugation at 500×g and incubated for 10 min. in 100 μl of ice-cold 50 mM Tris pH 8.0 supplemented with 0.1%/vol SDS, 5 mM EDTA+protease inhibitors (see above). Chromatin was sheared by sonication to the point that 800-1000 bp fragments are produced (titration data not shown). All samples were subjected to centrifugation for 10 min. at 6,700×g and supernatants containing DNA-protein fragments were transferred into new tubes and mixed with 400 μl of dilution buffer (50 mM Tris pH 8.0, 0.5%/vol NP40, 0.2M NaCl, 0.5 mM EDTA+protease inhibitors). To reduce the nonspecific background, all samples were pre-cleared for 30 min. with 80 μl of salmon sperm DNA-protein-A agarose slurry. Protein A-agarose beads (Sigma) were collected by brief centrifugation and 80 μl of each sample (input control) was set aside. For evaluation of IL-8 promoter the rest of the material was divided into two portions and incubated overnight with 4 μg each of α-p65NF-κB or α□-AP1 antibodies (Santa Cruz Biotechnology, Inc.); Immune complexes were collected by salmon sperm DNA-protein-A sepharose beads after 1 hour of incubation followed by 3 washes in high salt buffer (20 mM Tris pH 8.0, 0.1%/vol SDS, 1%/vol NP40, 2 mM EDTA, 0.5M NaCl), three washes with 1× TE buffer and elution with 1× TE supplemented with 2%/vol SDS. Eluted samples were heated at 65° C. for 10 min. Formaldehyde cross-links were reversed in all samples (including input control) by adding NaCl to final concentration of 0.3M and incubation at 65° C. for 5 hours. C

Chromatin associated protein was digested for 1 hour with 100 μg/ml Proteinase K in the presence of 10 mM EDTA and 20 mM Tris-Cl pH 6.5 at 45° C. DNA samples were extracted with pheno/chloroform and precipitated with 95%/vol ETOH. DNA concentration was calculated based on the OD reading measured by spectrophotometer at wavelength of 260 nm; all samples were subjected to the 40 cycles of amplification using respective pairs of primers flanking the NF-κB binding site.

The qRT-PCR detection of NF-κB binding DNA templates was performed using TaqMan Gene Expression Assay on 7300 real-time PCR System (Applied Biosystems). Briefly; triplicates of total reaction mixture of 25 μl were composed of DNA standardized by input control, 12.5 μl of 2×ABI TaqMan PCR Master Mix, and 0.25 μl (90 μM) of both sense and antisense primers (synthesized by Invitrogen) and 0.5 μl (10 μM) of the probe (synthesized by Applied Biosystems). After the initial denaturation at 95° C. (10 min), target genes were amplified through 40 cycles of universal cycling conditions (95° C./10s, 60° C./1 min). Standard dilution curves were calibrated using serial dilutions of control plasmid pTA_LTR representing 1×10⁶, 1×10⁵, 1×10⁴, 1×10³, 1×10² and 10 copies of DNA per reaction and all values are expressed as means±standard deviation of the mean. The qRT-PCR data for each experimental system were divided by data for their respective input control providing the numerical value (arbitrary units) representing the extent of LTR promoters available for p65 NF-κB binding.

As shown in FIG. 10, the amount of LTR complexes precipitated by antibody to X-DING-CD4 was higher than complexes precipitated by antibody to p65NF-kappaB. Since the X-DING-CD4 protein does not bind directly to LTR, but it does bind to NF-kappaB, the complexes pulled down by antibody to X-DING-CD4 could only be the X-DING-CD4/NF-kappaB/LTR. The increased amount of these X-DING-CD4/NF-kappaB/LTR complexes suggested that in the presence of X-DING-CD4 phosphatase, the LTR is blocked by dephosphorylation of NF-kappaB bound to HIV promoter. Dephosphorylated NF-kappaB is inactive to initiate transcription of LTR.

These results suggest that the endogenous X-DING-CD4 protein binds to NF-κB/LTR complex.

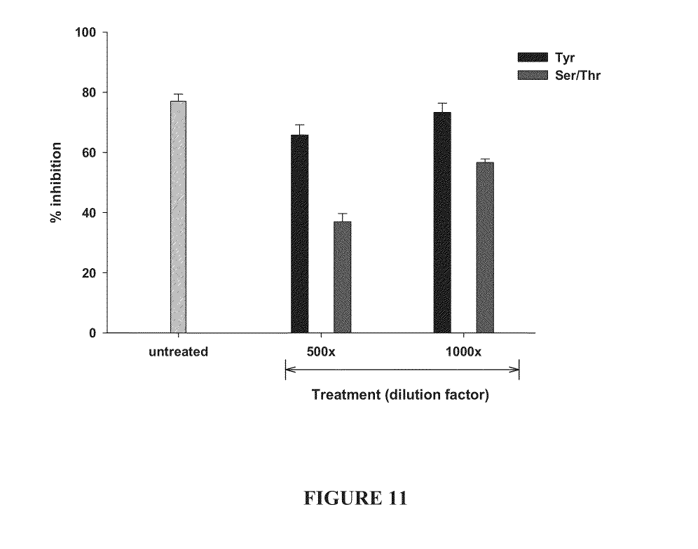

Once NF-κB/LTR-X-DING complex forms, the X-DING-CD4 dephosphorylates the NF-κB (steps 7-9), leading to NF-κB's dissociation from the LTR. The phosphatase enzymatic activity of X-DING-CD4 protein was measured by rapid suppression assay (RSA) similar to that described in Example 2. As shown in FIG. 11, samples treated with serine/threonine but not tyrosine phosphatase inhibitor had reduced activity to block HIV-1 LTR transcription. This result suggested that X-DING-CD4 protein is a serine/threonine specific phosphatase.

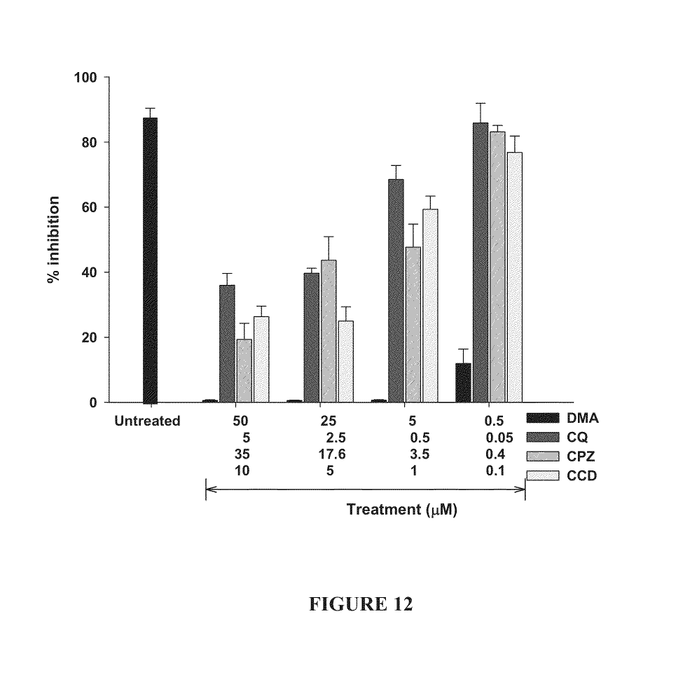

The cellular uptake of sX-DING does not require cell surface receptor but is mediated by macropinocytosis (step 1). Cellular uptake of X-DING-CD4 protein was tested by RSA in 1G5 cells treated with chemicals affecting: (i) clatrin mediated endocytosis—Chromopromazine (CPZ); (ii) endocytosis—cytochalasin D (CCD) and chloroquine (CQ) or (iii) macropinocytosis—dimethyl amiloride (DMA). Untreated group referred to the experimental system where X-DING-CD4 mediated inhibition of transcription was measured without cellular exposure to chemicals. As shown in FIG. 12, cells treated with DMA abolished the inhibitory effect of sX-DING-CD4 on LTR transcription. In contrast, cells treated with CQ, CPZ or CCD still demonstrated inhibitory effects of sX-DING-CD4 on LTR transcription.

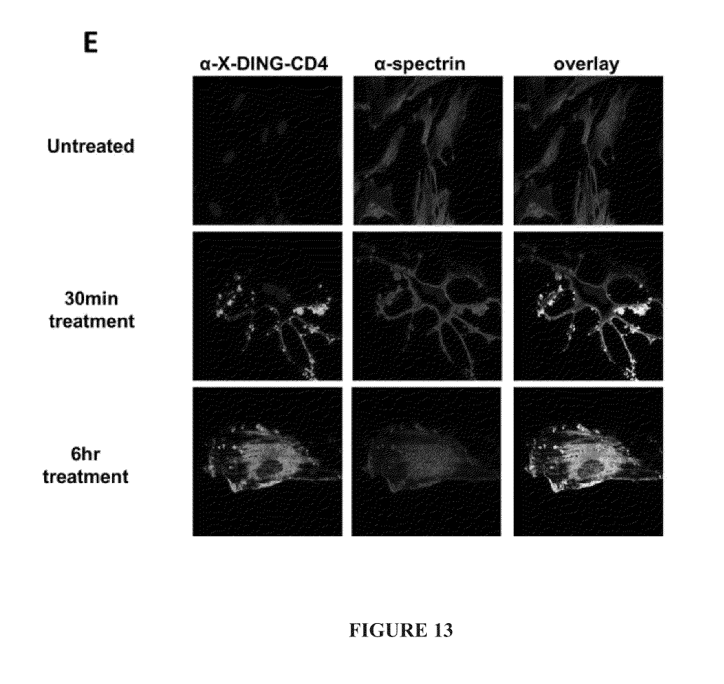

The cellular uptake of exogenous sX-DING-CD4 could also be visualized in cells. In this example, human fetal astrocytes cultured on cover slips were exposed to 100 ng/ml sX-DING-CD4 protein. Thirty minutes or 6 hours later cells were washed thoroughly with PBS and fixed for staining. The intracellular proteins were blotted with antibodies to X-DING-CD4 (green) and Spectrin (red) based on standard protocols. Images were taken by confocal microscopy. As shown in FIG. 13, in the untreated cells, no X-DING-CD4 could be detected. In cells treated with sX-DING-CD4 protein for 30 minutes, X-DING-CD4 could be detected on cell membranes. In contrast, in cells treated with sX-DING-CD4 protein for 6 hours, X-DING-CD4 could be detected on cell membrane, in cytosol, and in nucleus.

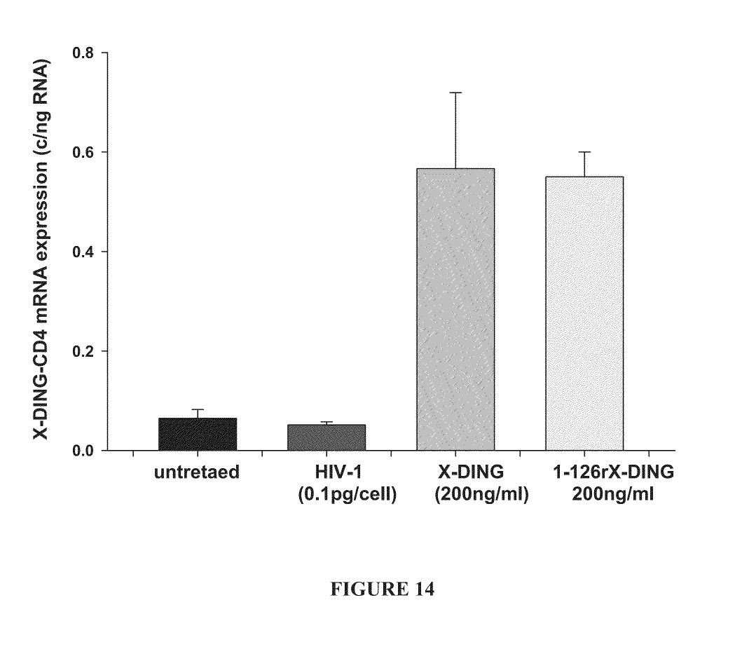

The sX-DING-CD4 may be released from the macropinosome to endoplasmic reticulum (ER) and cause induction of IFNα, which in turn induces the expression of X-DING-CD4 mRNA and endogenous X-DING-CD4 protein (step 2). The induction of X-DING-CD4 mRNA by sX-DING-CD4 was tested in 1G5 cells. Briefly, three groups of 1G5 cells were treated for 6 hours with NL4-3 HIV-1 at MOI of 0.1, 200 ng/ml native X-DING-CD4 protein, and 1-126 rX-DING-CD4 peptide (i.e., rX-DING-CD4^{pep}), respectively. As shown in FIG. 14, the untreated cells show level of constitutive X-DING-CD4 mRNA expression in 1G5 cells. HIV-1 exposure reduced the constitutive expression of this gene, while both X-DING-CD4 and 1-126 rX-DING-CD4 peptide (i.e., rX-DING-CD4^{pep}) induced expression of X-DING-CD4 mRNA. This result is consistent with our finding that IFN-α protein induces the expression of X-DING-CD4 gene (Sachdeva, 2013). The eX-DING-CD4, derived from the newly synthesized X-DING-CD4, binds CN and is transported into the nucleus where it blocks LTR transcription (steps 2-7).

In line with the mechanism in FIG. 7, rX-DING-CD4^{pep} and its synthetic peptide derivatives may permeate the cell membrane through macropinocytosis similar to how native X-DING-CD4 protein permeates the cell membrane. The subsequent effect of this cellular uptake is activation of the ER and IFN-α followed by induction of X-DING-CD4 mRNA and production of endogenous X-DING-CD4 protein. Therefore, therapeutic effects of blocking HIV-1 LTR transcription (step 7) can be achieved by a fragment of X-DING-CD4 protein (short X-DING-CD4 peptides) either alone or in combinations.

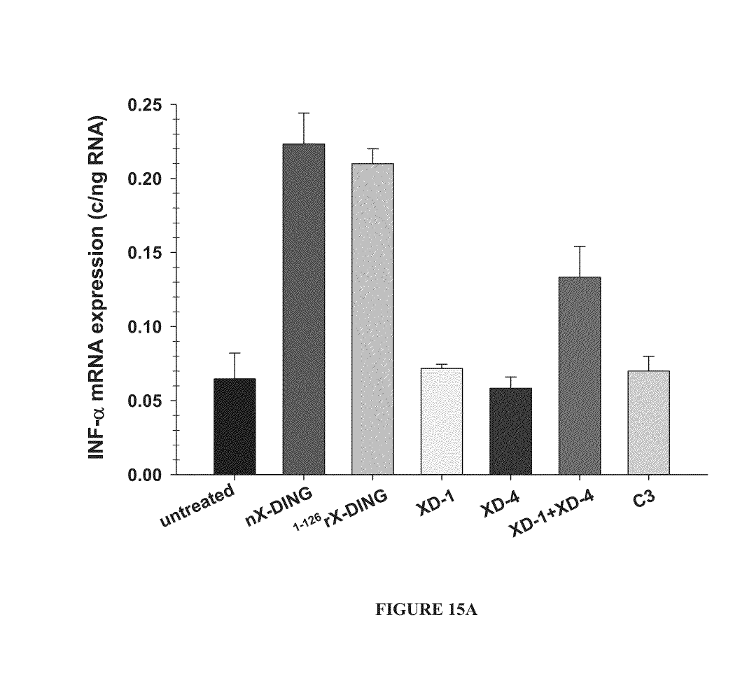

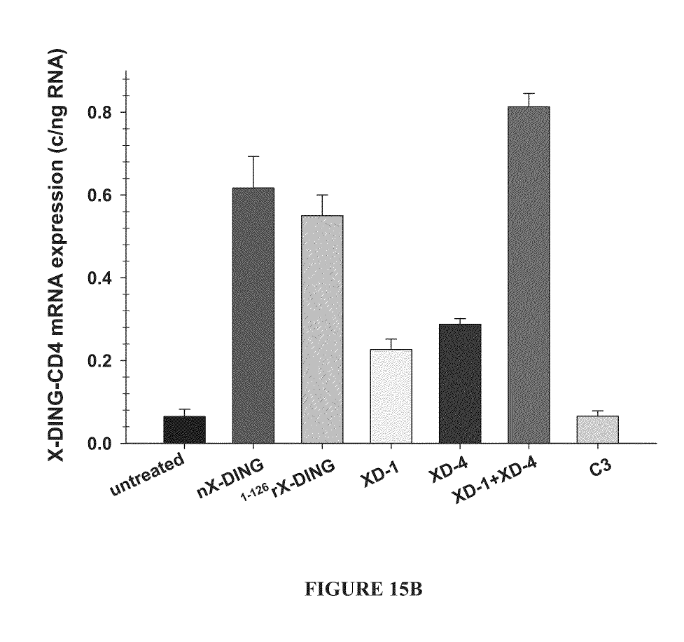

For example, we found that synthetic X-DING-CD4 peptides induced innate immunity responses similar to native X-DING-CD4 or X-DING-CD4^{pep}. Two peptide fragments from the N-terminal region of X-DING-CD4 were synthesized and their activities in inducing IFN-α and X-DING-CD4 mRNA expressions were determined by qRT-PCR. The concentration of proteins used in the assays was 200 ng/ml except for the combination of XD-1 and XD-4 where we used 100 ng/ml of each peptide. The 1-126 rX-DING-CD4 and peptide C3 p16 served as positive and negative controls, respectively. The C3 Peptide P16 was derived from the C3d component of serum complement. C3 Peptide P16 regulates B cells, but not T cells, through interacting with the gp140 C3d receptor (CR2). As shown in FIGS. 15A and 15B, a combination of XD-1 and XD-4 peptides induced expression of both IFN-α and X-DING-CD4 genes. The result suggests that the interaction of X-DING-CD4 synthetic peptides with the endoplasmic reticulum (ER) initiates this novel innate immunity response to HIV infection.

In one embodiment, such peptide may be connected or otherwise linked to cholesterol particles so that the peptide can enter cells through the native pathway of X-DING-CD4 cellular entry and intracellular release from the macropinosome. In another embodiment, such peptide alone or in combination with one another can enter cells through the native pathway of X-DING-CD4 cellular entry and intracellular release from the macropinosome. An advantage of the X-DING-CD4 peptide-based therapy is that such delivery ensures an escape from activation of adverse or otherwise harmful immune responses.

### Example 7

X-DING-CD4 Exhibits More Potent Anti-HIV-1 Activities than HPBP. PDING and PfluDING

We compared the anti-HIV-1 activities of four DING family members: PfluDING from Pseudomonas fluorescens, pDING from Hypericum perforatum, and human phosphate binding protein (HPBP), X-DING-CD4. The detailed information referring to the isolation and purification of X-DING-CD4, HPBP and pDING was previously described (Darbinian-Sarkissian et al., 2006; Lesner et al., 2009; Morales et al., 2006; Scott and Wu, 2005). PfluDING protein was used in the form of a site-specific mutation (S32G), altering one of the phosphate-binding residues, and resulting in a protein purified by the same method as the wild-type protein (Scott and Wu, 2005). For evaluations of the biological activity, all four proteins were dialyzed against 10 mM Tris-HCl, pH 8.0 using benzoylated cellulose tubing with an molecular weight cut-off of 1.2 kDa (Sigma). Subsequently the dialyzed material was concentrated by lyophilization and stored at 4° C.

TABLE 2
The comparison of the IC50 values acquired from
dose versus response evaluations in MAGI and RSA.

IC₅₀ (μg/ml)

| Treatment | MAGI | RSA |
|---|---|---|
| X-DING-CD4 | 0.075 | 0.052 |
| HPBP | 0.150 | 0.449 |
| pDING | 0.101 | 0.160 |
| PfluDING-S32G | 0.311 | 0.254 |

We performed the rapid suppression assay (RSA) which tests directly the inhibition of HIV-1 transcription (similar to the procedure described in Example 2), the MAGI assay which tests the inhibition of HIV-1 replication in a single cycle infection (similar to the procedure described in Example 2), and the HIV-1 infection inhibition assay in human PBLs.

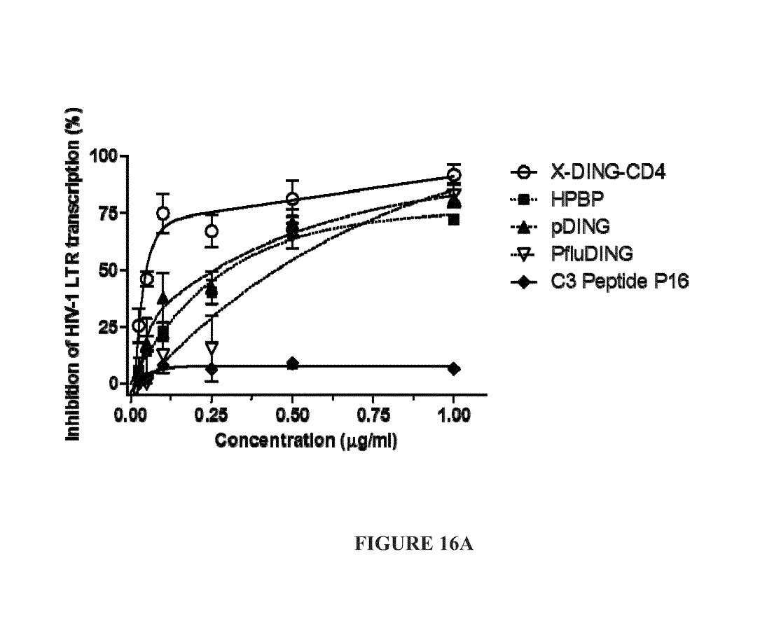

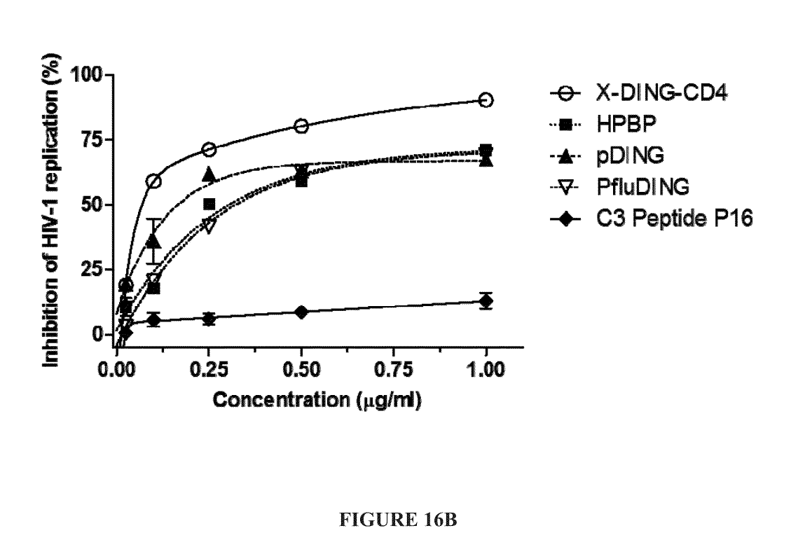

As shown in FIG. 16A, the HIV-1 LTR IC₅₀ for X-DING-CD4, HPBP, pDING and PfluDING was 52 ng/ml, 449 ng/ml, 160 ng/ml and 254 ng/ml, respectively. Treatment of cells with control C3 Peptide P16 had only a minor effect on HIV-1 LTR activity, blocking its expression by 4-9%.

Similar to the inhibition of HIV-1 LTR transcription, the MAGI assay showed that replication of HIV-1 was also blocked to various degrees. As shown in FIG. 16B, the IC₅₀ values for X-DING-CD4, HPBP, pDING and PfluDING was 75 ng/ml, 150 ng/ml, 101 ng/ml and 311 ng/ml, respectively. The control C3 Peptide P16 had only a minor effect on HIV-1 replication, with 11% inhibition at the highest dose of 1 m/ml.

The RSA and MAGI data reflected the direct effect of these four proteins on HIV-1 LTR transcription and the replication of virus in a single-cycle infection. Based on the IC₅₀ values in both the RSA and MAGI assays (Table 2), it appeared that X-DING-CD4 was the most potent inhibitor of HIV-1 transcription and replication, followed by HPBP and pDING approximately at the same level and finally by PfluDING.

In addition, we measured the inhibitory activities of these four proteins in HIV-1-infected peripheral blood lymphocytes (PBLs). The cells are treated separately with the proteins and the replication of virus was measured five and seven days later by assessing the intracellular levels of p24 core protein. PBLs were obtained by elutriation from the whole blood of healthy, HIV-1 negative volunteers. Before the experiment, cells were stimulated for 2 days in Dulbecco's modified Eagle medium (DMEM) supplemented with 10% fetal bovine serum (FBS), phytohemaglutinin (PHA, 5 μg/ml; Sigma), interleukin 2 (10 U/ml; R&D Systems), antibiotics and glutamate. Subsequently, cells were cultured without PHA. Briefly, 3×10⁶ PBLs/well in a 24-well plate (Costar Scientific) were cultured in 1 ml RPMI medium supplemented with antibiotics, glutamate and 1 μg/ml of each protein, respectively. Six hours after the initial exposure to the protein treatments, the culture medium was supplemented with 3%/Vol FBS. One day after treatment, cells were infected with 0.01 pg/cell NL4-3 HIV-1 isolate (Adachi et al., 1986) and cultured as described above, except that the concentration of FBS was adjusted to 5%/Vol. The experimental control consisted of HIV-1-infected but untreated PBLs. Replication of virus was evaluated at five and seven days after infection by Elisa assay of the intracellular HIV-1 p24 core antigen (Perkin Elmer). The viability of cells was assessed by the dye exclusion method (Strober, 2001) at 1, 2, 3, 5 and 7 days after protein treatments.

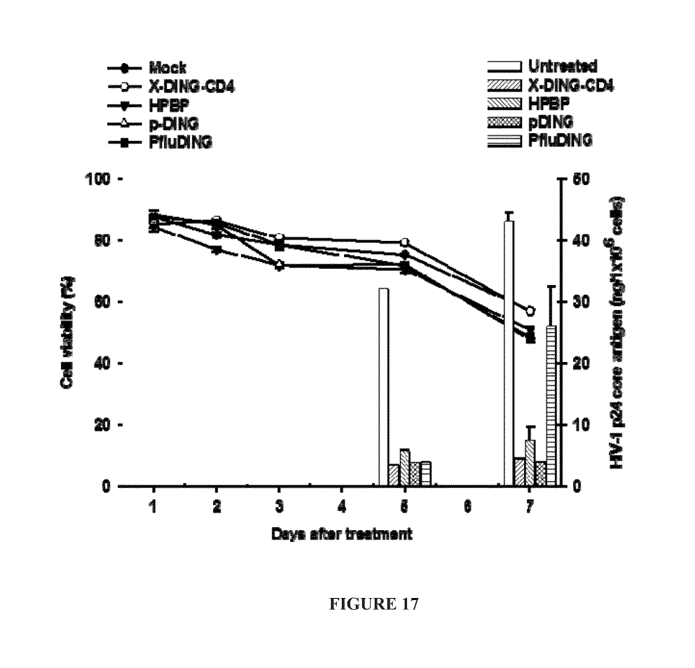

As shown in FIG. 17, five days after infection, the intracellular p24 core protein was lower by 6 to 10-fold in protein-treated samples as compared to the untreated control. Seven days after infection, the X-DING-CD4 and pDING treatments reduced replication of HIV-1 by about 11-fold, HPBP by about 6-fold and PfluDING by about 1.7-fold. See the right Y-axis and the bars. Throughout the course of this experiment, the viability of cells treated with proteins was comparable to the untreated sample, thus alleviating concerns of treatment-induced cytotoxicity. See the left Y-axis and the lines. Therefore, X-DING-CD4 exhibited similar inhibitory effects to pDING but more potent inhibitory effects than HPBP and PfluDING in this HIV infection inhibition assay in PBLs.

While the present invention has been disclosed with reference to certain embodiments, numerous modifications, alterations, and changes to the described embodiments are possible without departing from the full scope of the invention, as described in the specification and claims.

### REFERENCES

Adachi, A., H. E. Gendelman, S. Koenig, T. Folks, R. Willey, A. Rabson, and M. A. Martin. 1986. Production of acquired immunodeficiency syndrome-associated retrovirus in human and nonhuman cells transfected with an infectious molecular clone. J. Virol. 59:284-91.
Adams, L., S. Davey, and K. Scott. 2002. The DING protein: an autocrine growth-stimulatory protein related to the human synovial stimulatory protein. Biochim Biophys Acta. 1586:254-64.
Aguilar-Cordova, E., J. Chinen, L. Donehower, D. E. Lewis, and J. W. Belmont. 1994. A sensitive reporter cell line for HIV-1 tat activity, HIV-1 inhibitors, and T cell activation effects. AIDS Res Hum Retroviruses. 10:295-301.
Amini, S., N. Merabova, K. Khalili, and N. Darbinian. 2009. p38SJ, a novel DINGG protein protects neuronal cells from alcohol induced injury and death. J Cell Physiol. 221:499-504.
Belenky, M., J. Prasain, H. Kim, and S. Barnes. 2003. DING, a genistein target in human breast cancer: a protein without a gene. J Nutr. 133:2497 S-2501S.
Berna, A., F. Bernier, E. Chabriere, T. Perera, and K. Scott. 2007. DING proteins; novel members of a prokaryotic phosphate-binding protein superfamily which extends into the eukaryotic kingdom. Int J Biochem Cell Biol.
Berna, A., F. Bernier, K. Scott, and B. Stuhlmuller. 2002. Ring up the curtain on DING proteins. FEBS Lett. 524:6-10.
Berna, A., K. Scott, E. Chabriere, and F. Bernier. 2009. The DING family of proteins: ubiquitous in eukaryotes, but where are the genes? Bioessays. 31:570-80.
Bernier, F., and A. Berna. 2001. Germins and germin-like proteins: plant do-all proteins. But what do they do exactly? Plant Physiol. Biochem. 39:545-554.
Bush, D., H. Fritz, C. Knight, J. Mount, and K. Scott. 1998. A hirudin-sensitive, growth-related proteinase from human fibroblasts. Biol. Chem. 379:225-9.
Chackerian, B., E. M. Long, P. A. Luciw, and J. Overbaugh. 1997. Human immunodeficiency virus type 1 coreceptors participate in postentry stages in the virus replication cycle and function in simian immunodeficiency virus infection. J Virol. 71:3932-9.
Cherrier, T., M. Elias, A. Jeudy, G. Gotthard, V. Le Douce, H. Hallay, P. Masson, A. Janossy, E. Candolfi, O. Rohr, E. Chabriere, and C. Schwartz. 2011. Human-Phosphate-Binding-Protein inhibits HIV-1 gene transcription and replication. Virol J. 8:352.
Collombet, J. M., M. Elias, G. Gotthard, E. Four, F. Renault, A. Joffre, D. Baubichon, D. Rochu, and E. Chabriere. 2010. Eukaryotic DING proteins are endogenous: an immunohistological study in mouse tissues. PLoS ONE. 5:e9099.
Darbinian-Sarkissian, N., A. Darbinyan, J. Otte, S. Radhakrishnan, B. E. Sawaya, A. Arzumanyan, G. Chipitsyna, Y. Popov, J. Rappaport, S. Amini, and K. Khalili. 2006. p27(SJ), a novel protein in St John's Wort, that suppresses expression of HIV-1 genome. Gene Ther. 13:288-95.
Darbinian, N., M. Czernik, A. Darbinyan, M. Elias, E. Chabriere, S. Bonasu, K. Khalili, and S. Amini. 2009. Evidence for phosphatase activity of p27SJ and its impact on the cell cycle. J Cell Biochem. 107:400-7.
Diemer, H., M. Elias, F. Renault, D. Rochu, C. Contreras-Martel, C. Schaeffer, A. Van Dorsselaer, and E. Chabriere. 2008. Tandem use of X-ray crystallography and mass spectrometry to obtain ab initio the complete and exact amino acids sequence of HPBP, a human 38-kDa apolipoprotein. Proteins. 71:1708-20.
Falasca, K., M. R. Manigrasso, D. Racciatti, P. Zingariello, M. Dalessandro, C. Ucciferri, P. Mancino, M. Marinopiccoli, C. Petrarca, P. Conti, E. Pizzigallo, M. T. Guagnano, and J. Vecchiet. 2006. Associations between hypertriglyceridemia and serum ghrelin, adiponectin, and IL-18 levels in HIV-infected patients. Ann Clin Lab Sci. 36:59-66.
Hadas, E., A. Borjabad, W. Chao, M. Saini, K. Ichiyama, M. J. Potash, and d. and Volsky. 2007. Testing antiretroviral drug efficacy in conventional mice infected with chimeric HIV-1. AIDS. 21:1-5.
Hain, N. A., B. Stuhlmuller, G. R. Hahn, J. R. Kalden, R. Deutzmann, and G. R. Burmester. 1996. Biochemical characterization and microsequencing of a 205-kDa synovial protein stimulatory for T cells and reactive with rheumatoid factor containing sera. J Immunol. 157:1773-80.
Hatada, E. N., D. Krappmann, and C. Scheidereit. 2000. NF-kappaB and the innate immune response. Curr Opin Immunol. 12:52-8.
Hayouka, Z., A. Levin, M. Maes, E. Hadas, D. E. Shalev, D. J. Volsky, A. Loyter, and A. Friedler. 2010. Mechanism of action of the HIV-1 integrase inhibitory peptide LEDGF 361-370. Biochem Biophys Res Commun. 394:260-5.
Ivanova, A., R. Y. Shilpi, R. Sachdeva, G. Li, and M. Simm. 2012. Native X-DING-CD4 protein secreted by HIV-1 resistant CD4+ T cells blocks activity of IL-8 promoter in human endothelial cells infected with enteric bacteria. Innate Immun. 18:571-9.
Lesner, A., A. Kartvelishvili, J. Lesniak, D. Nikolov, M. Kartvelishvili, G. Trillo-Pazos, E. Zablotna, and M. Simm. 2004. Monoubiquitinated histone H1B is required for antiviral protection in CD4(+)T cells resistant to HIV-1. Biochemistry. 43:16203-11.
Lesner, A., Y. Li, J. Nitkiewicz, G. Li, A. Kartvelishvili, M. Kartvelishvili, and M. Simm. 2005. A soluble factor secreted by an HIV-1-resistant cell line blocks transcription through inactivating the DNA-binding capacity of the NF-kappa B p65/p50 dimer. J Immunol. 175:2548-54.
Lesner, A., R. Shilpi, A. Ivanova, M. A. Gawinowicz, J. Lesniak, D. Nikolov, and M. Simm. 2009. Identification of X-DING-CD4, a new member of human DING protein family that is secreted by HIV-1 resistant CD4(+) T cells and has anti-viral activity. Biochem Biophys Res Commun. 389:284-9.
Li, G., S. Aaron, K. Kazmierczak, A. Lesner, Y. Li, A. Ivanova, G. Bentsman, M. J. Potash, and M. Simm. 2007 Inhibition of HIV-1 or bacterial activation of macrophages by products of HIV-1-resistant human cells. Immunol Cell Biol. 85:603-9.
Meyer, J. M., A. Ji, L. Cai, and D. R. van der Westhuyzen. 2012. High-capacity selective uptake of cholesteryl ester from native LDL during macrophage foam cell formation. J Lipid Res. 53:2081-91.
Morales, R., A. Berna, P. Carpentier, C. Contreras-Martel, F. Renault, M. Nicodeme, M. L. Chesne-Seck, F. Bernier, J. Dupuy, C. Schaeffer, H. Diemer, A. Van-Dorsselaer, J. C. Fontecilla-Camps, P. Masson, D. Rochu, and E. Chabriere. 2006. Serendipitous discovery and X-ray structure of a human phosphate binding apolipoprotein. Structure. 14:601-9.
Pahl, H. L. 1999. Activators and target genes of Rel/NF-kappaB transcription factors. Oncogene. 18:6853-66.
Potash, M. J., W. Chao, G. Bentsman, N. Paris, M. Saini, J. Nitkiewicz, P. Belem, L. Sharer, A. I. Brooks, and D. J. Volsky. 2005. A mouse model for study of systemic HIV-1 infection, antiviral immune responses, and neuroinvasiveness. Proc Natl Acad Sci USA. 102:3760-5.
Renault, F., E. Chabriere, J. P. Andrieu, B. Dublet, P. Masson, and D. Rochu. 2006. Tandem purification of two HDL-associated partner proteins in human plasma, paraoxonase (PON1) and phosphate binding protein (HPBP) using hydroxyapatite chromatography. J Chromatogr B Analyt Technol Biomed Life Sci. 836:15-21.
Roshorm, Y., J. P. Hong, N. Kobayashi, A. J. McMichael, D. J. Volsky, M. J. Potash, M. Takiguchi, and T. Hanke. 2009. Novel HIV-1 clade B candidate vaccines designed for HLA-B*5101(+) patients protected mice against chimaeric ecotropic HIV-1 challenge. Eur J Immunol. 39:1831-40.
Sachdeva, R., Shilpi, Y. R., and Simm, M. 2013. The interplay between the X-DING-CD4, IFN-alpha and IL-8 gene activity in quiescent and mitogen or HIV-1 activated PBMCs from elite controllers, AIDS progressors and HIV(−) controls. Innate Immun. In press.
Saini, M., E. Hadas, D. J. Volsky, and M. J. Potash. 2007. Vaccine-induced protection from infection of mice by chimeric human immunodeficiency virus type 1, EcoHIV/NL4-3. Vaccine. 25:8660-3.
Scott, K., and L. Wu. 2005. Functional properties of a recombinant bacterial DING protein: comparison with a homologous human protein. Biochim Biophys Acta. 1744:234-44.
Simm, M., L. S. Miller, H. G. Durkin, M. Allen, W. Chao, A. Lesner, M. J. Potash, and D. J. Volsky. 2002. Induction of secreted human immunodeficiency virus type 1 (HIV-1) resistance factors in CD4-positive T lymphocytes by attenuated HIV-1 infection. Virology. 294:1-12.
Strober, W. 2001. Trypan blue exclusion test of cell viability. Curr Protoc Immunol. Appendix 3:Appendix 3B.
Tamalet, C., J. Fantini, C. Tourres, and N. Yahi. 2003. Resistance of HIV-1 to multiple antiretroviral drugs in France: a 6-year survey (1997-2002) based on an analysis of over 7000 genotypes. AIDS. 17:2383-8.

What we claim are:

1. A pharmaceutical composition for preventing or treating a pathological condition in a subject, comprising a pharmaceutically effective amount of:
a. an X-DING-CD4 peptide having an amino acid sequence set forth in SEQ ID NO: 25;
b. a derivative of the X-DING-CD4 peptide; or
c. a combination thereof.

2. The pharmaceutical composition of claim 1, wherein the pathological condition is a viral infection.

3. The pharmaceutical composition of claim 2, wherein the viral infection is a retroviral infection.

4. The pharmaceutical composition of claim 3, wherein the retroviral infection is a HIV virus infection.

5. The pharmaceutical composition of claim 4, wherein the HIV virus is HIV-1.

6. The pharmaceutical composition of claim 1, wherein the derivative is selected from the group consisting of polypeptides having amino acid sequences set forth in SEQ ID NOS: 1-15.

7. The pharmaceutical composition of claim 1, wherein the therapeutically active derivative of the X-DING-CD4 peptide has at least about 85% sequence identity to the X-DING-CD4 peptide.

8. The pharmaceutical composition of claim 1, wherein the pharmaceutically effective amount is from about 1 nM to about 200 nM, in particular from about 8 nM to about 150 nM, preferentially 100 nM.

9. The pharmaceutical composition of claim 1, wherein the subject is a mammal.

10. The pharmaceutical composition of claim 9, wherein the mammal is a human.

11. A method for preventing or treating a pathological condition in a subject, comprising the administration of a pharmaceutically effective amount of:
a. an X-DING-CD4 peptide having an amino acid sequence set forth in SEQ ID NO: 25;
b. a derivative of the X-DING-CD4 peptide; or
c. a combination thereof.

12. The method of claim 11, wherein the pathological condition is a viral infection.

13. The method of claim 12, wherein the viral infection is a retroviral infection.

14. The method of claim 13, wherein the retroviral infection is a HIV virus infection.

15. The method of claim 14, wherein the HIV virus is HIV-1.

16. The method of claim 11, wherein the derivative is selected from the group consisting of polypeptides having amino acid sequences set forth in SEQ ID NOS: 1-15.

17. The method of claim 11, wherein the therapeutically active derivative of the X-DING-CD4 peptide has at least about 85% sequence identity to the X-DING-CD4 peptide.

18. The method of claim 11, wherein the pharmaceutically effective amount is from about 1 nM to about 200 nM, in particular from about 8 nM to about 150 nM, preferentially 100 nM.

19. The method of claim 11, wherein the subject is a mammal.

20. The method of claim 19, wherein the mammal is a human.

21. An isolated cDNA, encoding an X-DING-CD4 peptide comprising the amino acid sequence set forth in SEQ ID NO: 25 or a derivative of the X-DING-CD4 peptide.

22. The isolated cDNA of claim 21, wherein the derivative is selected from the group consisting of polypeptides having amino acid sequences set forth in SEQ ID NOS: 1-15.

23. The isolated cDNA of claim 21, where the polynucleotide is cloned into a vector to form a plasmid, wherein the plasmid is optionally contained in a host cell.

24. The isolated cDNA of claim 25, wherein the plasmid vector is pET-28a and the host is a bacteria E. coli BL21.

25. The isolated cDNA of claim 23, wherein the plasmid vector is pcDNA 3.1 vector and the host is a mammalian cell.

26. The isolated cDNA of claim 25, wherein the mammalian cell is Human Embryonic Kidney 293T cells.

27. An isolated cDNA comprising the nucleic acid sequence set forth in SEQ ID NOs: 16 or 26.

28. An isolated cDNA that is complementary to the polynucleotide of claim 21.

29. An isolated cDNA that is complementary to the polynucleotide of claim 27.

30. An isolated polypeptide, selected from the group consisting of:
a. a polypeptide comprising an amino acid sequence having at least 85% identity to SEQ ID NO: 25; and
b. a derivative of the polypeptide in (a).

31. The isolated polypeptide of claim 30, wherein the glutamic acid at position 68 in SEQ ID No: 25 is methylated.

32. The isolated polypeptide of claim 30, wherein the polypeptide derivative is selected from the group consisting of polypeptides having amino acid sequences set forth in SEQ ID NOS: 1-15.

33. The isolated polypeptide of claim 30, wherein the isolated polypeptide blocks a NF-κB-mediated biological pathway.

34. The isolated polypeptide of claim 33, wherein the NF-κB-mediated biological pathway controls a viral gene transcription.

35. The isolated polypeptide of claim 34, wherein the viral gene transcription is a HIV-1 LTR gene transcription.

36. The isolated polypeptide of claim 33, wherein the NF-κB-mediated biological pathway controls an inflammatory reaction.

37. The isolated polypeptide of claim 36, wherein the inflammatory reaction is a lipopolysaccharide-induced inflammatory reaction.

38. The isolated polypeptide of claim 37, wherein the lipopolysaccharide is from a bacteria.

39. The isolated polypeptide of claim 38, wherein the bacteria is a Salmonella typhimurium, Shigella flexneri, or Camplylobacter jejuni.

40. The isolated polypeptide of claim 30, wherein the isolated polypeptide inhibits a viral infection.

41. The isolated polypeptide of claim 40, wherein the viral infection is a HIV infection.

42. The isolated polypeptide of claim 30, wherein the isolated polypeptide inhibits an inflammatory reaction.

43. The isolated polypeptide of claim 42, wherein the inflammatory reaction is a lipopolysaccharide-induced inflammatory reaction.

44. The isolated polypeptide of claim 43, wherein the lipopolysaccharide is from a bacteria.

45. The isolated polypeptide of claim 44, wherein the bacteria is a Salmonella typhimurium, Shigella flexneri, or Camplylobacter jejuni.

46. A process for producing a polypeptide comprising culturing the host cell of claim 23 under conditions sufficient for the production of the polypeptide.

47. The process of claim 46, further comprising recovering the polypeptide so produced.

48. The process of claim 47, wherein the protein is recovered using ion-exchange chromatography.

49. The process of claim 48, wherein the ion-exchange chromatography is a Ni-affinity column.

50. A composition for a cell-based therapy, comprising a cDNA encoding an X-DING-CD4 peptide having an amino acid sequence set forth in SEQ ID NO: 25, or a derivative of the X-DING-CD4 peptide.

51. The composition of claim 50, wherein the cDNA is inserted in cell-base therapy vector.

52. The composition of claim 51, wherein the vector is retroviral vector or an adenovirus vector.

53. The composition of claim 51, wherein the cell-based therapy is a therapy to prevent or treat viral infection.

54. The composition of claim 53, wherein the viral infection is a HIV infection.

55. The composition of claim 53, wherein the cell-based therapy is a therapy to prevent or treat inflammation reactions.

56. A method of a cell-based therapy, comprising administrating into a cell a cDNA encoding an X-DING-CD4 peptide having an amino acid sequence set forth in SEQ ID NO: 25, a derivative of the X-DING-CD4 peptide, or a combination thereof.

57. The method of claim 56, wherein the cDNA is inserted in cell-base therapy vector.

58. The method of claim 57, wherein the vector is retroviral vector or an adenovirus vector.

59. The method of claim 56, wherein the cell-based therapy is a therapy to prevent or treat viral infection.

60. The method of claim 59, wherein the viral infection is a HIV infection.

61. The method of claim 56, wherein the cell-based therapy is a therapy to prevent or treat inflammation reactions.


SEQUENCE LISTING (28 sequences)

SEQ ID NO: 1 - protein, 126 aa, Homo sapiens
MADINGGGATLPQPLYQTSGVLTAGFAPYIGVGSGKGKLAFLNNDYSQFGTGTKNVHWAGSDSKLTSTELSTYASTKQAAWGKLIQVPSVATSVAIPFNKAGSNAVDLSVDQLCGVFSGRITTWNQ

SEQ ID NO: 2 - protein, 28 aa, Artificial Sequence
  synthetic
GTGTKNVHWAGSDSKLTATELSTYATDK

SEQ ID NO: 3 - protein, 26 aa, Artificial Sequence
  synthetic
LAFLNNDYSQEGTGTKNVHWAGSDSK

SEQ ID NO: 4 - protein, 11 aa, Artificial Sequence
  synthetic
DINGGQTLPQK

SEQ ID NO: 5 - protein, 23 aa, Artificial Sequence
  synthetic
GTKNVHWAGSDSKLTSTELSTYC

SEQ ID NO: 6 - protein, 21 aa, Artificial Sequence
  synthetic
GTKNVHWACVSKLTSTELSTY

SEQ ID NO: 7 - protein, 24 aa, Artificial Sequence
  synthetic
GVSNKNVHWAGSDSKLTATELSTY

SEQ ID NO: 8 - protein, 5 aa, Artificial Sequence
  synthetic
YTSLE

SEQ ID NO: 9 - protein, 24 aa, Artificial Sequence
  synthetic
GVSNKNVHWAGSDSKLTATQLSTY

SEQ ID NO: 10 - protein, 5 aa, Artificial Sequence
  synthetic
QLSTY

SEQ ID NO: 11 - protein, 19 aa, Artificial Sequence
  synthetic
GVSNKNVHWAGSDSKLTAT

SEQ ID NO: 12 - protein, 24 aa, Artificial Sequence
  synthetic
GVSNKNVHWAGSDSKLTATLLSTY

SEQ ID NO: 13 - protein, 5 aa, Artificial Sequence
  synthetic
LLSTY

SEQ ID NO: 14 - protein, 12 aa, Artificial Sequence
  synthetic
DINGGGATLPQK

SEQ ID NO: 15 - protein, 20 aa, Artificial Sequence
  synthetic
KNVHWAGSDSKLTSTELSTY

SEQ ID NO: 16 - DNA, 379 bp, Homo sapiens
atggcagacatcaacggcggcggcgccaccctgccacaaccgctgtaccagacctccggcgtattgacagccggtttcgccccgtacatcggtgtgggcagcggcaaaggcaaactggcgttcctgaacaacgactacagccagttcggcaccggcaccaagaacgtgcactgggcgggcagcgactccaagctgacctcgactgaactgtccacctacgcctccaccaaacaagcggcctggggcaagctgattcaagtgccttcggtagccacttcggttgccattccattcaacaaggccggttccaacgcggttgacctgagcgttgatcaactgtgcggcgtgttctcgggccgcatcaccacctggaaccaac

SEQ ID NO: 17 - DNA, 38 bp, Artificial Sequence
  synthetic
tcgttcagcttggtcatcacgccttggctggtcacggc

SEQ ID NO: 18 - DNA, 39 bp, Artificial Sequence
  synthetic
gcgcgcggatccgccatggcagacatcaacggcggcggc

SEQ ID NO: 19 - DNA, 35 bp, Artificial Sequence
  synthetic
cgcggcaagctttcctggctgaagatcaggttggt

SEQ ID NO: 20 - protein, 7 aa, Artificial Sequence
  synthetic
YGRKKRR

SEQ ID NO: 21 - protein, 11 aa, Artificial Sequence
  synthetic
YGRKKRRQRRR

SEQ ID NO: 22 - protein, 5 aa, Artificial Sequence
  synthetic
RRQRR

SEQ ID NO: 23 - DNA, 24 bp, Artificial Sequence
  synthetic
tttgcagaactacacaccagggcc

SEQ ID NO: 24 - DNA, 24 bp, Artificial Sequence
  synthetic
ctccctggaaagtccccagcggaa

SEQ ID NO: 25 - protein, 292 aa, Homo sapiens
MADINGGGATLPQPLYQTSGVLTAGFAPYIGVGSGKGKLAFLNNDYSQFGTGTKNVHWAGSDSKLTSTELSTYASTKQAAWGKLIQVPSVATSVAIPFNKAGSNAVDLSVDQLCGVFSGRITTWNQLPATGRTGNIVVVYRNEASGTTELFTRFLTAKCVNESKKFVVTTNFADSFGVPAGAVPAVTSQGVMTKLNAGDGRITYMSPDYAAPTLAGLDDATKVAKVAGVSPAPDNVSAAIAAVPVPAAANVALQNAWVPVFAAAADANDPSVVPYPSTGFPILGFTNLIFSQ

SEQ ID NO: 26 - DNA, 879 bp, Homo sapiens
gccatggcagacatcaacggcggcggcgccaccctgccacaaccgctgtaccagacctccggcgtattgacagccggtttcgccccgtacatcggtgtgggcagcggcaaaggcaaactggcgttcctgaacaacgactacagccagttcggcaccggcaccaagaacgtgcactgggcgggcagcgactccaagctgacctcgactgaactgtccacctacgcctccaccaaacaagcggcctggggcaagctgattcaagtgccttcggtagccacttcggttgccattccattcaacaaggccggttccaacgcggttgacctgagcgttgatcaactgtgcggcgtgttctcgggccgcatcaccacctggaaccaactcccggctaccggtcgcaccggtaacatcgtggtggtttaccgtaacgaagccagtggcaccaccgagctgttcacccgtttcctgacagccaagtgcgtcaatgagtccaagaagtttgtggtcaccaccaactttgcagacagcttcggcgtgccggcaggtgccgtgcctgccgtgaccagccaaggcgtgatgaccaagctgaacgccggtgatggtcgcatcacttacatgagcccggactatgccgctcctaccttggctggcctggatgacgccaccaaagtggccaaggtcgccggtgtttctccagcccctgacaacgtttccgctgccattgctgctgtgccagtaccggcggctgccaacgtcgccctgcagaacgcctgggtaccagtatttgctgctgctgccgacgccaatgacccaagcgtcgtgccttacccaagcaccggctttccaatcctgggcttcaccaacctgatcttcagccag

SEQ ID NO: 27 - protein, 9 aa, Artificial Sequence
  synthetic
ELFTRFLTA

SEQ ID NO: 28 - DNA, 43 bp, Artificial Sequence
  synthetic
cgcggcaagctttcgttcagcttggtcatcacgccttggctgg